(19) United States
(12) Patent Application Publication
(10) Pub. No.: US 2009/0176776 A1
(43) Pub. Date: Jul. 9, 2009

(54) SMALL MOLECULE INHIBITORS OF HIV-1 CAPSID ASSEMBLY

(75) Inventor: Peter Prevelige, Birmingham, AL (US)

Correspondence Address: NIXON PEABODY, LLP, 401 9TH STREET, NW, SUITE 900, WASHINGTON, DC, 20004-2128, US

(73) Assignee: UNIVERSITY OF ALABAMA AT BIRMINGHAM, Birmingham, AL (US)

(21) Appl. No.: 12/090,361

(22) PCT Filed: Oct. 23, 2006
(86) PCT No.: PCT/US2006/041284

§ 371 Date: Sep. 24, 2008

Related U.S. Application Data
(60) Provisional application No. 60/728,797, filed on Oct. 21, 2005.

(51) Int. Cl. A61K 31/5377; A61K 31/506; A61P 31/18

(52) U.S. Cl. 514/231.8; 514/256

(57) ABSTRACT

The present invention provides novel methods of treating HIV infections employing small molecule inhibitors identified by chemical library (DIVERSet™ library). These small molecule inhibitors may specifically bind to HIV-1 capsid protein thereby interfering with capsid assembly. The small molecule inhibitors of the present invention can be potential drug targets in the treatment of HIV infection.

### CROSS REFERENCE TO RELATED APPLICATIONS

This application claims priority to U.S. Patent Application Ser. No. 60/728,797, which is incorporated herein in its entirety.

### GOVERNMENT RIGHTS

The United States Government has rights to this invention under Grant No. NIH AI44626, granted by the National Institute of Health.

### TECHNICAL FIELD OF THE INVENTION

This invention relates generally to methods of treating HIV infections in mammals, including humans. More particularly, the invention relates to the use of chemical compounds, referred herein as “small molecule inhibitors,” for the treatment of HIV infection in subjects through the inhibition of HIV-1 capsid assembly.

### BACKGROUND OF THE INVENTION

In the twenty-five years since AIDS was first described, much has been learned about the effects of HIV-mediated disease and the drugs used to treat it. There is still no cure, however, and current antiretroviral drugs cause major, and sometimes fatal, side effects. An effective vaccine looms in the future. Assembly inhibitors are one of several new drug approaches to therapeutic agents for the treatment of HIV/AIDS.

Researchers and drug manufacturers have developed an effective way to treat HIV/AIDS patients by using a combined therapeutic use of reverse transcriptase inhibitors and protease inhibitors. Such therapy is known as HAART (highly active antiretroviral therapy). Although the use of HAART has effectively reduced the number of ADS-related deaths, this alone fails to suppress the virus indefinitely. Additionally, new HAART-related medical complications have been reported in HIV/AIDS patients. The complications include nausea, vomiting, reduction of red or white blood cells and metabolic changes such as abnormal fat distribution, abnormal lipid and glucose metabolism and bone loss, peripheral neuropathy, and mitochondrial toxicity. See, e.g., HIV Infection and AIDS: An Overview, available on line at the NIAID website at niaid.nih.gov/factsheets/hivinf.htm).

Beside the above complications, further viral infections readily lead to drug-resistant mutants and latent forms of persistent infections. These resistant mutants can be maintained in sites that are not susceptible to current drugs. Indeed, close to 50% of patients fail to efficiently suppress viral replication on treatment mainly due to resistance issues and tolerability/compliance of current drug regimens. Moreover, the current drug regimens are costly and dosing guidelines are complicated to follow. Thus, additional HIV therapies are urgently required.

### SUMMARY OF THE INVENTION

A feature of the present invention provides an approach to inhibiting HIV replication by targeting the cellular and viral components that are involved in HIV-1 capsid assembly and maturation. By inhibiting the functions and interactions between viral Gag protein components, the viral particles fail to assemble and mature correctly, leaving them immature and noninfectious. Included in this invention is the discovery of methods for administering therapeutically effective amount of small molecule inhibitors that inhibit the formation of HIV-1 capsid assembly.

According to the invention, there is also provided a pharmaceutical composition for the treatment of HIV infection, comprising a therapeutically effective amount of a compound of a small molecule inhibitor, a pharmaceutically acceptable salt thereof, or a pharmaceutically effective prodrug thereof and a pharmaceutically acceptable carrier.

Another aspect of the present invention is directed to methods for administering therapeutically effective amount of a small molecule inhibitor that inhibits the formation of HIV-1 capsid assembly in the treatment of HIV infection.

This method comprises the administration of a pharmaceutically effective amount of a small molecule inhibitor and a pharmaceutically acceptable carrier for treating a human suffering HIV infection. According to the invention, small molecule inhibitors exhibit therapeutic properties and are useful in the treatment of HIV infection.

According to the invention, there is provided a method for treating HIV infection comprising administering a small molecule inhibitor in combination with a therapeutically effective agent selected from the group consisting of chemotherapeutic agents, anti-retroviral inhibitors, cytokines, hydroxyurea, monoclonal antibodies that bind to the GAG proteins.

### BRIEF DESCRIPTION OF THE DRAWINGS

The drawings are provided for illustration, not limitation.

FIG. 1 shows the lifecycle of HIV.

FIG. 2 shows the targets of inhibition in the HIV lifecycle of the invention.

FIG. 3 shows the structure of HIV virus in the assembly and maturation stages.

FIG. 4 are the pictures of infectivity of immature and mature virions by Cryo-Electron microscopy (EM).

FIG. 5 is a graphical representation of optical density results showing the polymerization of capsid assembly.

FIG. 6 shows thin section EM analysis of polymerized capsid (CA) protein formed with dilution technique.

FIG. 7 is a picture of capsid protein with spherical and cylindrical hexamer lattice by EM.

FIG. 8 shows a picture of three site interaction during assembly.

FIG. 9 shows a graphical representation of C:C domain interaction in the capsid protein.

FIG. 10 shows the C-domain inhibits capsid assembly.

FIGS. 11A-E illustrate examples of the chemical compounds with the molecular weight, the chemical structure and IC₅₀ data identified according to the invention.

FIG. 12 shows the toxicity assay results of the compounds. The figure represents the reduction of the cell numbers versus the concentration of the compounds compared to different cell lines.

FIG. 13 shows the anti-HIV efficacy and toxicity assays for the compound 26: (A) Jurkat cells assay (B) Luc-M7 assay and (C) 293 T cell assay.

FIG. 14 shows the anti-HIV efficacy and toxicity assays for the compound 41: (A) Jurkat cells assay and (B) Luc-M7 assay.

FIG. 15 shows the anti-HIV efficacy and toxicity assays for the compound 58: (A) Jurkat cells assay (B) Luc-M7 assay and (C) 293 T cell assay.

FIG. 16 shows the anti-HIV efficacy and toxicity assays for the compound 59: (A) Jurkat cells assay and (B) Luc-M7 assay.

FIG. 17 shows the anti-HU efficacy and toxicity assays for the compound 60: (A) Jurkat cells assay and (B) Luc-M7 assay.

FIG. 18 shows the anti-HU efficacy and toxicity assays for the compound 70: (A) Jurkat cells assay (B) Luc-M7 assay and (C) 293 T cell assay.

FIG. 19 shows the Saturation Transfer Difference NMR (STD-NMR) Spectroscopy to demonstrate that compound 26 binds to both the N-terminal domain as well as C-terminal domain of the CA protein: (A) the reference ID-NMR spectrum of the low-field region of a solution of compound 26; (B) the STD-NMR spectrum of the compound 26; (C) the STD-NMR spectrum of the compound 26 with C-terminal domain of the CA protein, and (D) the STD-NMR spectrum of the compound 26 with N-terminal domain of the CA protein.

### DETAILED DESCRIPTION OF THE INVENTION

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms “a,” “an,” and “the” include the plural reference unless the context clearly indicates otherwise. Thus, for example, the reference to an excipient is a reference to one or more such excipients, including equivalents thereof known to those skilled in the art. Other than in the operating examples, or where otherwise indicated, all numbers expressing quantities of ingredients or reaction conditions used herein should be understood as modified in all instances by the term “about.” The term “about” when used in connection with percentages may mean±1%.

All patents and other publications identified are incorporated herein by reference for the purpose of describing and disclosing, for example, the methodologies described in such publications that might be used in connection with the present invention, but are not to provide definitions of terms inconsistent with those presented herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains.

HIV-1 (and retroviruses in general) assembles through the controlled polymerization of the Gag polyprotein. The Gag polyprotein is transported to the plasma membrane and forms patches within which assembly occurs (Weldon, R. A., Jr., and E. Hunter. 1997, Molecular Requirements for Retrovirus Assembly, in Structural Biology of Viruses, Oxford University Press, New York, 1997, pp. 381-410). Transport to the plasma membrane acts to concentrate the Gag protein but is apparently not essential as Gag protein expressed at sufficiently high levels is capable of cytoplasmic assembly (Boulanger and Jones, Curr. Topics Microbiol. Immunol., 214:237-260, 1996). During assembly, the patches enlarge and bud outward ultimately pinching off from the cell. During budding, the virion acquires the envelope proteins necessary for receptor binding, as well as a lipid envelope.

Morphologically, the released immature virion presents as an enveloped particle approximately 100 nm in diameter, containing a spherical immature core (Nermut and Hockley, Curr. Topics Microbiol. Immunol., 214:1-24, 1996). The viral protease is incorporated into the virion as part of a Gag-Pol fusion protein, arising from a minus one (−1)-translational frameshift. Budding activates the protease, which cleaves the Gag polyprotein into the matrix (MA), capsid (CA), and nucleocapsid (C) domains as well as the ‘spacer’ peptides P2, P1, and the C-terminal P6 domain. The immature virion is metastable, and cleavage of the polyprotein is associated with a profound morphological change in the virion (Vogt, Curr. Topics Microbiol. Immunol., 214:95-131, 1996). The matrix domain stays associated with the membrane envelope, the capsid domain collapses to form a conical core, and the nucleocapsid domain condenses with the viral RNA in the center of the conical capsid core (mature virion). The structural rearrangements necessarily arise from the disruption of existing interdomain contacts and the formation of new ones.

Formation of the mature viral core is a crucial step in the life cycle of the virus. Many mutations that block or lead to the formation of cores have been reported to inhibit viral infectivity. The capsid is composed of many subunits which form several homo- or hetero-polymers of protein. The noncovalent bonds between capsomeres in a viral assembly stabilize a folded protein domain. The interface between two subunits can look very much like a single domain, with amino acid side chains tightly packed against one another. A common feature to most of the virus structures analyzed is the way in which a polypeptide chain from one subunit can extend under or over domains of neighboring subunit. These extended polypeptide arms intertwine with other polypeptide arms and help to stabilize the capsid by initiating hydrophobic interactions, hydrogen bonding, and salt bridges. Contacts between individual unit, and for some viruses also contacts with core proteins, determine the overall capsid structure. Repeated contacts can often occur and the resulting structure is symmetrical.

Viral self assembly is driven by the stability of the interactions between protein subunits under conditions that favor association. Although the capsids of many viruses differ in protein composition, a general viral structural design has evolved characterized by polymerized subunits that, in turn, are composed of several homo- or hetero-polymers of protein. The nucleocapsid is asymmetrical having a long dimension of about 100 nm, a wide free end about 40-60 nm, and a narrow end about 20 nm in width. The nucleocapsid within each mature virion is composed of two molecules of the viral single-stranded RNA genome encapsulated by proteins proteolytically processed from the Gag precursor polypeptide. Cleavage of the gag gene polyprotein by a viral coded protease (PR) produces mature capsid proteins. These gag gene products are the matrix protein (p17) that is thought to be located between the nucleocapsid and the virion envelope; the major capsid protein (p24), that forms the capsid shell; and the nucleocapsid protein (p9) that binds to the viral RNA genome. This proteolytic processing in infected cells is linked to virion morphogenesis.

The major capsid protein p24 (also called CA) contains about 240 amino acids and exhibits a molecular weight of 24-27 kD. The protein p24 self-associates to form dimers and oligomeric complexes as large as dodecamers. Genetic studies with mutations in the HIV-1 Gag polyprotein have identified several functional domains in the p24 protein including the C terminal half of the molecule and a major homology region (MHR) spanning 20 amino acids that is conserved in the p24 proteins of diverse retroviruses. These mutations appear to affect precursor nucleocapsid assembly.

The nature of the large scale motions within the virion required for protease access, maturation, and uncoating has not been well-characterized. There are two reasons why this is so: the enveloped, non-icosahedral, polymorphic nature of the virion makes crystallographic analysis of the intact virion nearly impossible, and techniques to study the dynamics of large protein complexes have not been readily available.

To further understand the structure, stability, and intersubunit interfaces between the various domains of the Gag polyprotein, the present inventors developed a high-resolution mass spectrophotometry to measure hydrogen-deuterium exchange profiles of the Gag domains, which in turn, can compare the exchange rates of different morphological forms of Gag protein (e.g., monomeric and dimeric capsid protein, spherical and cylindrical capsid polymers, CA-NC monomers and CA-NC polymers with RNA and individual Gag domains compared to the intact polyprotein). See U.S. Patent Application Publication No. 2001/0036627 A1, (Ser. No. 09/800,240, filed Mar. 6, 2001), which claims priority to U.S. Provisional Application Ser. No. 60/186,981, filed Mar. 6, 2000. The entire contents of these applications, including the specifications, claims and drawings are incorporated herein by reference in their entirety.

The CA protein can assemble into a curved sheet of interlocked hexameric units. Hexameric rings in an assembled capsid are formed by repetitive interactions of N domains, which in turn, are tethered together by the C domain. The CA molecules can dimerize through homotypic C domain interactions. However, the C terminal domains can inhibit assembly via formation of biologically inactive heterodimer (Lanman, J. et al., J. Virol., 76: 6900-6908, 2002; Prevelige, P., U.S. Patent Application Publication No. 2001/0036627 A1). Thus, data obtained from the study of the CA assembly can be used to screen anti-viral drugs and provide insights to the development of viral assembly inhibitors for treating HIV infections.

Once inside the cell, HIV uses specific enzymes (e.g., reverse transcriptase, protease, and integrase) to survive. To date, the FDA has approved the use of a number of antiretroviral drugs that interfere with the virus' ability to utilize these enzymes. These drugs include reverse transcriptase (RT) inhibitors, protease inhibitors (PI) and fusion inhibitors. RT inhibitors interfere with reverse transcriptase that HIV needs to make copies of itself. There are two main types of RT inhibitors: (i) nucleoside/nucleotide RT inhibitors that provide faulty DNA building blocks and halting the DNA chain that the virus uses to make copies of itself, and (ii) non-nucleoside RT inhibitors that bind to RT preventing the virus to carry out it duplication function. On the other hand, protease inhibitors interfere with the protease enzyme that HIV uses to produce infective virus particles. Fusion inhibitors function by changing the shape of the gp41 envelope protein surrounding HIV and, therefore, can interfere with the virus' ability to fuse with and enter the host cell. Integrase inhibitors are also being studied, as are other viral inhibitors (e.g., Tat and Rev inhibitors). As noted previously, however, the current antiretroviral drug regimens have short comings, leaving a need for additional approaches to antiretroviral therapies.

The term “treatment” or “treating” means any treatment of HIV infection including preventing, reducing, or curing clinical symptoms.

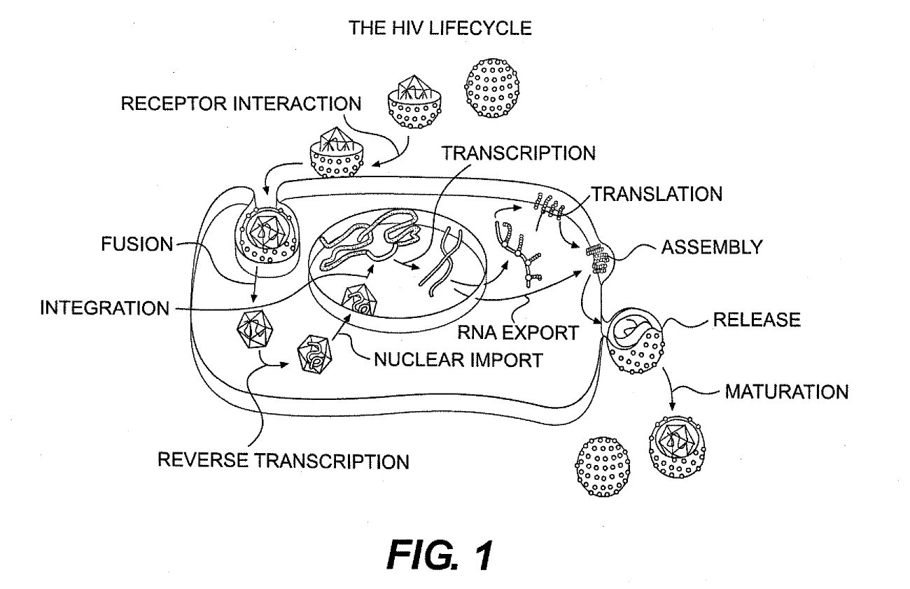

The lifecycle of HIV is shown in FIG. 1. Upon receptor interaction, HIV fuses to the host membranes and viral membranes, and enters into the cytoplasm. The viral lipid envelope is left behind in the host's lipid bilayer and the viral capsid is released into the cell. Immediately after entry, reverse transcriptase transcribes the viral genome into cDNA. The reverse transcriptase activity degrades the viral RNA template, and cDNA travels to the nucleus. Viral integrase proteins insert the viral genome into the host's chromosomal DNA, and cellular factors mediate transcription of viral transcription factor. Upon transcription, a regulatory protein binds a specific region on RNA and mediates the export from the nucleus. The polyprotein is transported to the plasma membrane and forms patches within which assembly occurs. During assembly, the patches enlarge and bud outward from the cell. The formation of the mature virion occurs after the structural rearrangements of the conical capsid core.

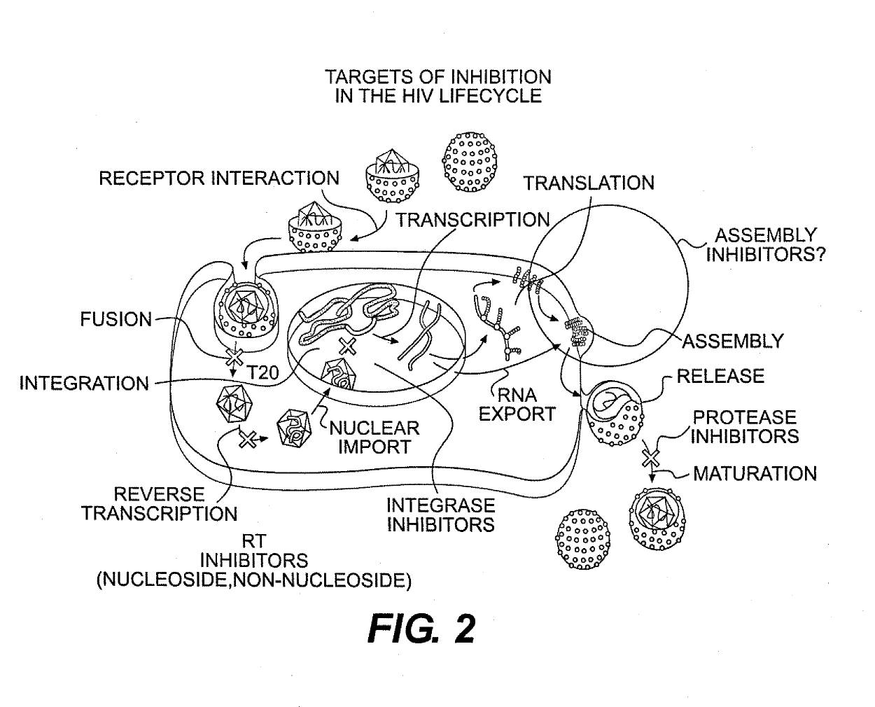

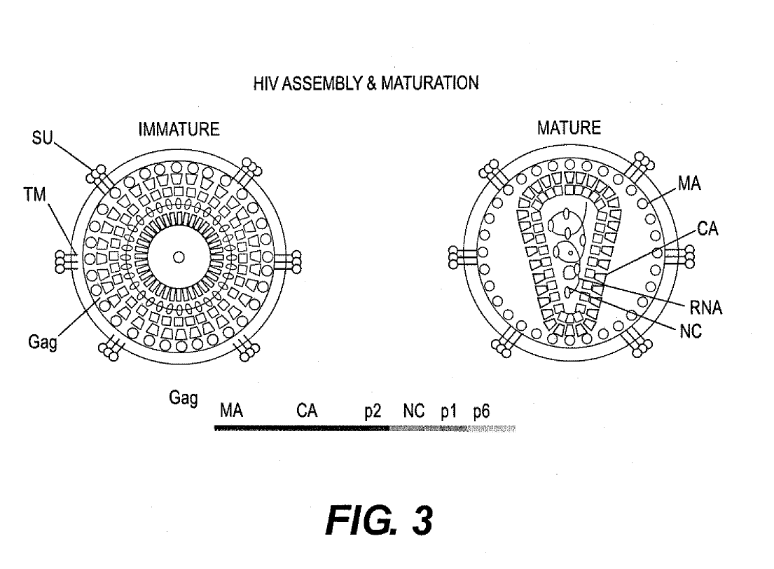

The invention relates to the use of certain chemical compounds, small molecule inhibitors, which have been found to inhibit the cellular and viral components that are involved in HIV-1 capsid assembly and maturation. The targets of inhibition in the HIV lifecycle are illustrated in FIG. 2. HIV assembles at the cell membrane through the polymerization of the GAG polyprotein as shown in FIG. 3. The Gag polyprotein consists of the MA, CA, and NC domain as well as the P2, P1, and P6 regions. Following assembly the virus particle buds from the cell to form immature viral particles. During budding the viral protease is activated which cleaves the Gag protein into its respective domains. This cleavage causes a profound morphological change in which the CA protein collapses away from the MA protein and forms a conical core. The MA remains associated with the membrane and the NC condenses with the viral genome. This maturation process is required for virus infectivity, and is the target of the antiviral drugs known as protease inhibitors. The maturation process it self represents an attractive target, but it may not be possible to use traditional structural techniques such as cryo-EM and X-ray crystallography to determine the structure of the viral particles because they are pleimorphic and non icosahedral.

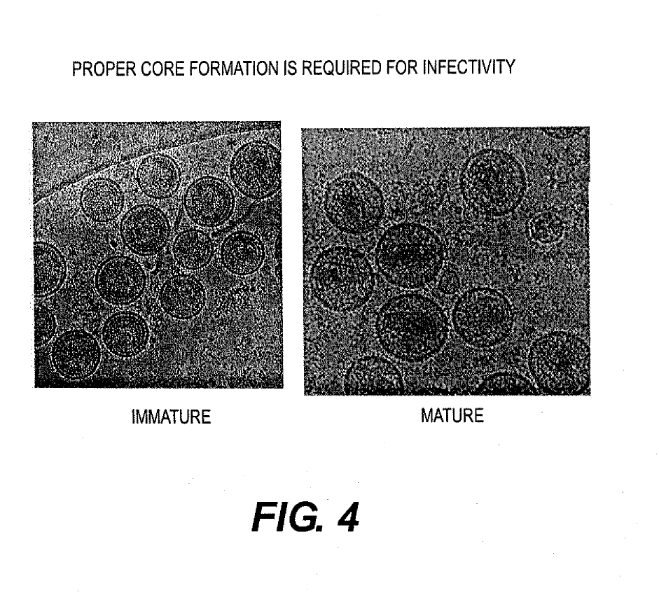

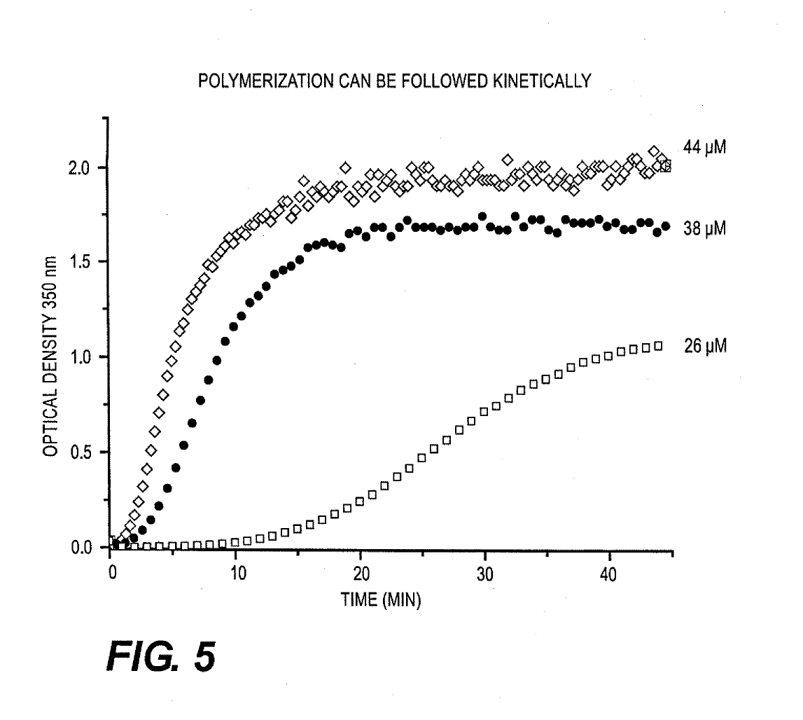

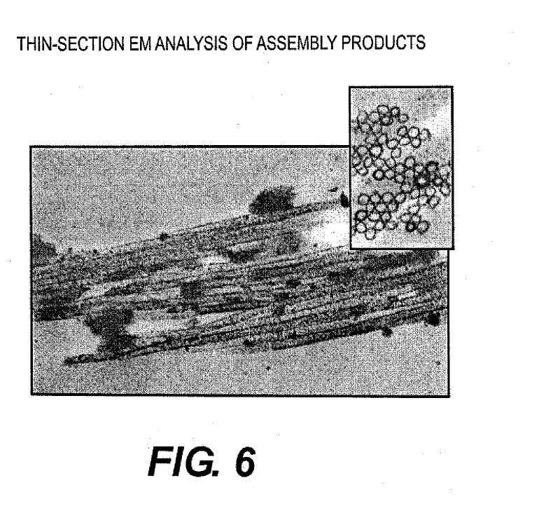

Cryo-EM methods have been used to study the structure of Gag polyprotein in immature virions and in virus-like particles. FIG. 4 shows that the spherical CA shells of the immature virions condense to form proper core formation in mature virions. The polymerization can be monitored kinetically by detecting the optical density as shown in FIG. 5. FIG. 6 is a example of thin section EM. for the tubes CA makes observed with. The tubes of CA from the side view and end are shown in FIG. 6. The interactions in these tube are believed to be similar to those observed in the conical core formed in the virus because a small amount of conical cores are formed in these assembly reactions. Thin section TEM showed that the polymerized capsid protein had formed cylinders similar to those previously observed. The inset show end on views of the approximately 33 nm diameter tubes. Thus, dilution into high salt produces biologically relevant structures.

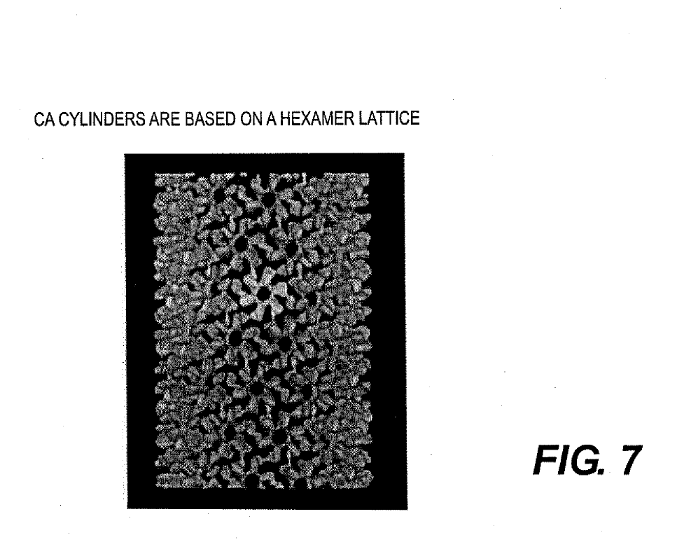

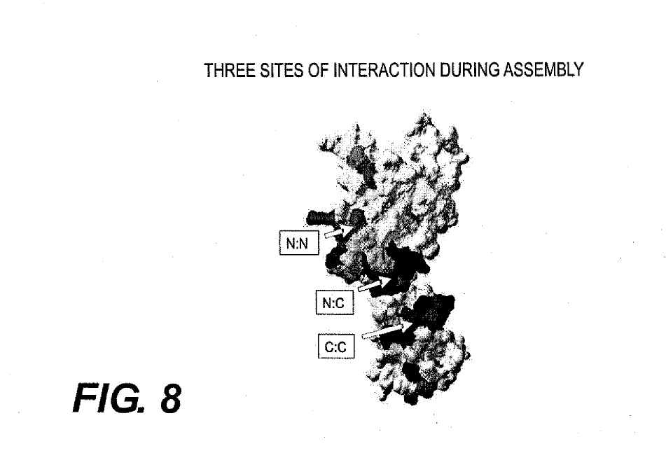

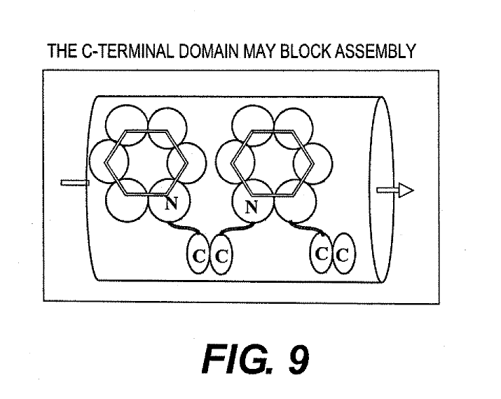

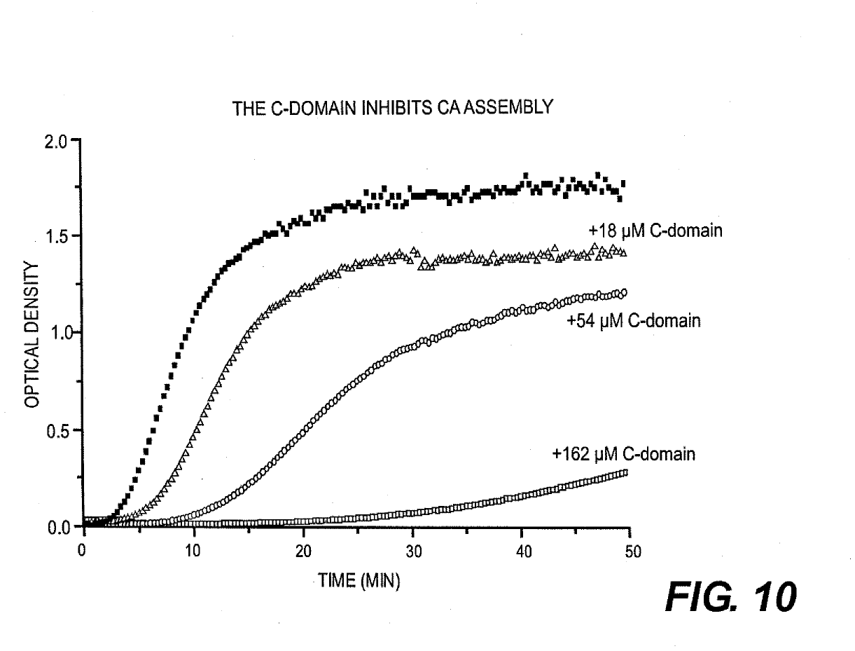

The left picture of FIG. 7 is the cryo-EM reconstruction of the CA tubes. The cryo-EM density of the tubes revealed hexamer subunits connected to an adjacent hexamer though a density at the ends of the hexamer lobes. The interactions are propagated to form a hexamer lattice that wraps around to form tubes. The N-domain structure was merged into the density of the hexamer lobes. The loop was oriented on the outside of the hexamer and the Helices 1 and 2 were positioned at the center of the hexamer. There are three binding sites of interaction during the assembly (FIG. 8). Hexameric rings in an assembled capsid are formed by repetitive interactions of N:N domain, N:C domain, and C:C domain. The CA molecules can dimerize through homotypic C domain interactions as illustrated in FIG. 9. The C-terminal domains can inhibit assembly via formation of biological inactive heterodimer. FIG. 10 shows that addition of the C-terminal domain of the HIV-1 Capsid to assembly reactions results in inhibition of assembly. This provides proof of principle for the ability of this assay to detect inhibitors of assembly. The kinetics can be followed by monitoring the increase of turbidity using a spectrophotometer. The IC₅₀ of the chemical compound tested is determined by titration. As the C-domain inhibits capsid assembly by forming CA-C-domain heterodimers, the effect of added C-terminal domain on the rate of assembly was evaluated by plotting the rate of assembly versus C-terminal domain concentration.

The invention relates to a method for inhibiting HIV replication in cells comprising administering to the cells an effective amount of a small molecule inhibitor of formulas I and II. The invention also relates to the a method for treating HIV administering, or the use of, a therapeutically effective amount of a small molecule inhibitor to inhibit the formation of HIV-1 capsid assembly in the treatment of HIV infection. It is discovered that certain small molecules inhibit capsid assembly by affecting intermolecular CA-CA interaction in the formation of HIV-1 capsid core particle during capsid assembly and/or by interfering with virus budding. The term “inhibition” refers to slowing, interrupting, arresting or stopping the condition and does not necessarily indicate a total elimination of the condition. It is believed that prolonging the survivability of a patient, beyond being a significant advantageous effect in and of itself, also indicates that the condition is beneficially controlled to some extent. The term IC₅₀ is the generally accepted measure of inhibition, refers to 50% of inhibitory concentration in competitive binding experiments and is well understood in the art.

### Small Molecule Inhibitors of HIV-1 Capsid Assembly

The small molecule inhibitors useful in the present invention are those compounds described by the structural formulas below. The small molecule inhibitors of formulas I, II, and III are effective inhibitors of HIV-1 capsid assembly. The compound may be prepared by standard organic synthesis. The compounds are also commercially available from ChemBidge Corp. (San Diego, Calif.) where they are members of the DIVERSet™ library of low molecular weight compounds. The compounds have bind to HIV capsid (CA) protein and, in turn, inhibit the assembly of the CA protein. When administering in suitable forms, the small molecule inhibitors described below are useful in the treatment of HIV infection. The DIVERSet™ library is a collection of chemical compounds which can be screened simultaneously (or if desired, sequentially) for a property of interest. As shown by the formulas below, the compounds found to inhibit HIV-1 capsid are related in structure and/or function.

For the compounds in formulas I, II, and III, the hydrocarbyl chains, e.g. alkyl, alkylene, alkenyl, alkynyl, etc., or the carbon chain of an alkoxy group may be straight chains or branched chains as is known in the art. For example, a C₁-C₆ alkyl group would include but not be limited to methyl, ethyl, n-propyl, i-propyl, n-butyl, i-butyl, sec-butyl, t-butyl, pentyl, neo-pentyl, hexyl and the like. Examples of alkoxy groups include methoxy, ethoxy, n-propoxy, n-butoxy, isopropoxy, isobutoxy, sec-butoxy, t-butoxy and the like.

A carbocyclic ring refers to cyclic hydrocarbon groups of 3 to 12 carbon atoms, which may be saturated unsaturated. The carbocylic ring is not limited to monocylic ring structures but may be a polycylic structure, e.g., a bicyclic, or tricyclic structure. For example, (C₃₋₇) cycloalkyl, either alone or in combination with another radical, means a cycloalkyl radical containing from three to seven carbon atoms. Exemplary carbocycles include, but are not limited to, cyclopropyl, cyclopentyl, cyclopentene, cyclopentadiene, cyclohexyl, cyclohexene, cyclohexadiene, cycloheptyl, cyclooctyl, bornane, bornene, norbornane, norpinane, adamantane, and the like.

The term “aryl” or “Ar” refers to aromatic hydrocarbon groups of 6 to 10 carbon atoms. For example, phenyl, either alone or in combination with another radical, means an aryl radical containing from six carbon atoms. Exemplary aryl groups include but are not limited to, phenyl, napthyl, or those resulting from antbracene, phenylene, phenathrene, pentalene, anzulen, indacene and other fused-ring structures.

The term “heterocycle” or “hetero” either alone or in combination with another radical, means a monovalent radical derived by removal of a hydrogen from a five-, six-, or seven-membered saturated or unsaturated (including aromatic) heterocycle containing from one to four heteroatoms selected from nitrogen, oxygen and sulfur. Furthermore, “hetero” as used herein, means a heterocycle as defined above fused to one or more other cyclic structure, be it a heterocycle or any other cyclic structure. Examples of suitable heterocycles and heteraryls include, but are not limited to pyrrolidine, furan, pyran, chromene, xantehene, thiazolidine, pyrrole, thiophene, diazfpine, imidazole, pyrazole, isoxazole, thiazole, tetrazole, piperidine, 1,4-dioxane, 4-morpholine, pyridine, pyrazine, pyrimidine, thiazolo[4,5-b]-pyridine, quinoline, isoquinoloine, quinuclidine, purine, indole, isoindole, indazole, and the like.

The term “halide” or “halogen” refers to fluoride, chloride, bromide, and iodide.

Various substituents within formulas I, II, and III may be substituted with any number of substituents or functional moieties. In general, the term “substituted” refers to the replacement of hydrogen radicals in a given structure with the radical of a specified substituent. When more than one position in any given structure may be substituted with more than one substituent selected from a specified group, the substituent may be either the same or different at every position. The term “substituted” is contemplated to include all permissible substituents of organic compounds. In a broad aspect, the permissible substituents include acyclic and cyclic, branched and unbranched, carbocyclic and heterocyclic, aromatic and non-aromatic, carbon and heteroatom substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment and prevention, for example of disorders, as described generally above. Examples of substituents include, but are not limited to, halo substituents, e.g. F; Cl; Br; or I; a hydroxyl group; a C₁-C₆ alkoxy group, e.g, —OCH₃, —OCH₂CH₃, or —OCH(CH₃)₂; a C₁-C₆ haloalkyl group, e.g., —CF₃; —CH₂CF₃; or —CHCl₂; C₁-C₆ alkylthio; amino; mono and dialkyl amino groups; —NO₂; —CN; ═O; ═S; a sulfate group, and the like. Additional examples of generally applicable substituents are illustrated by the specific compounds disclosed.

Compounds which may be used as small molecule inhibitors of HIV assembly according to the invention include compounds of formula I or pharmaceutically acceptable salts thereof:

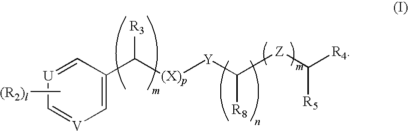

The various substituents and variables for compounds of formula I are defined as follows:

In formula (I), U is CR₁ or N and V is CR₁, or N. In preferred embodiments, both U and V or CR₁ or both U and V are N. More preferably, both U and V are CH or both U and V are N.

R₁ and R₂ are independently hydrogen, hydroxyl, halogen, carboxyl, nitro, —NH₂, —NHR₆, —NR₆R₇, substituted or unsubstituted C₁-C₆ alkyl, substituted or unsubstituted C₂-C₆ alkenyl, and substituted or unsubstituted C₂-C₆ alkynyl, substituted or unsubstituted C₁-C₆ alkoxyl group, C₃-C₇ alkylene forming a substituted or unsubstituted second ring on the ring containing U and V, or C₁-C₄ amide group forming a substituted or unsubstituted second ring on phenyl ring, an aliphatic or aromatic ring substituent selected from the group consisting of substituted or unsubstituted C₃-C₈ cycloalkyl, substituted or unsubstituted C₅-C₈ cycloalkenyl and aryl wherein said aliphatic or aromatic ring substituent may be substituted with one or more 5- or 6-membered aromatic or aliphatic heterocyclic groups.

l is 1, 2, or 3.

R₆ and R₇ are independently substituted or unsubstituted C₁-C₆ alkyl, substituted or unsubstituted C₄-C₆ cycloalkyl, or substituted or unsubstituted C₁-C₆ alkylene-C₄-C₆ cycloalkyl.

R₃ is hydrogen, hydroxyl, substituted or unsubstituted C₁-C₆ alkyl, a bond which forms a double bond with X when X is a nitrogen, together with Y and R₁ or R₂ form a substituted or unsubstituted 5- to 11-membered heterocyclic ring; or together with Y and R₈ form a substituted or unsubstituted 5- to 11-membered heterocyclic ring. The heterocyclic ring may be saturated, unsaturated or aromatic, contains carbon atoms and 1, 2, 3, or 4 heteroatoms independently selected from the group consisting of N, NH, O and S. It may also be a polycyclic or fused ring structure. The nitrogen and sulfur heteroatoms may optionally be oxidized. The heterocyclic ring may be attached at any heteroatom or carbon atom which results in a stable structure. Preferably, R₃ is selected from hydrogen, hydroxyl group, optionally substituted C₁-C₆ alkyl or a bond which forms a double bond with X when X is a nitrogen. Most preferably, R₃ is hydrogen, methyl, or a bond which forms a double bond with X when X is a nitrogen.

n is 0, 1, 2, 3, 4, 5, 6, 7, or 8.

m is 0 or 1.

p is 0 or 1.

X is SO₂, C═O, N, or NH.

Y is NH, N, O, S, NC═O, or substituted or unsubstituted phenylene.

Z is O, SO₂, C═O, NH, or S(O)₂NH.

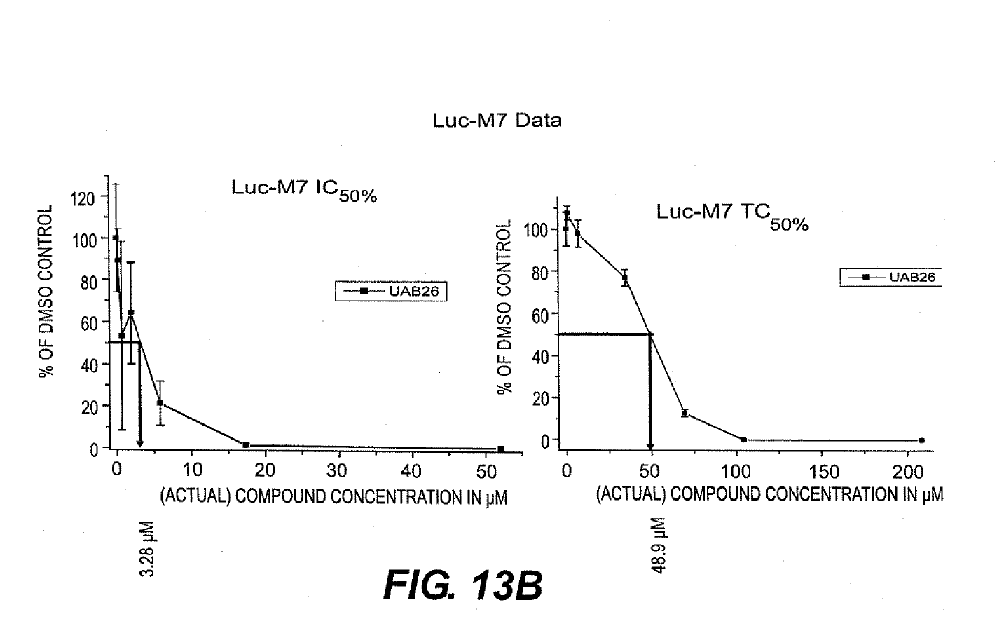

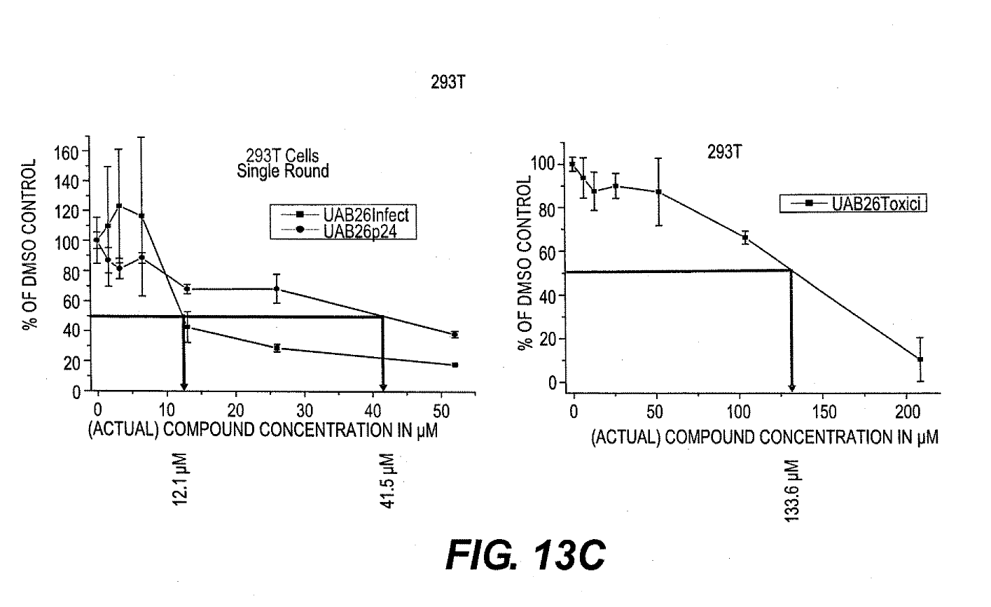

R₄ and R₅ are independently H, OH, SH, carboxyl, or together form a substituted or unsubstituted ring system selected from the group consisting of a C₃-C₁₁ carbocyclic ring system, a C₃-C₁₁ heterocyclic ring system, aryl, or heteroaryl. Preferably, R₄ and R₅ together form a substituted or unsubstituted ring system selected from phenyl, pyridyl, morpholinyl, imidazolyl, 1,2,3,4-tetrahydronaphthalenyl, and adamantyl. Preferred substituents for these ring systems include, hydroxyl, bromo, methoxy, diethylamino, carboxyl, nitro and combinations thereof. As shown by the compounds listed in FIG. 13, the rings preferably have 2, 3, or 4 such substituents.

R₈ is H, OH, SH, carboxyl, substituted or unsubstituted C₁-C₆ alkyl, or a substituted or unsubstituted ring system selected from the group consisting of a C₃-C₁₁ carbocyclic ring system, a C₃-C₁₁ heterocyclic ring system, aryl, or heteroaryl.

A preferred group of small molecule inhibitors which may be used in the invention include compounds of formula II:

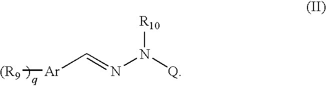

In formula II, Ar is a phenyl group or a pyridinyl group, which bear the substituent R₉. The structural formulas for R₉—Ar are shown below:

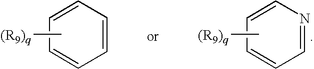

R₉ is independently a hydrogen, hydroxyl group, halogen, carboxyl, nitro oxide, C₁-C₆ alkoxyl group, C₃-C₇ alkylene group forming a second ring on Ar, or C₁-C₄ amide group forming a second ring on Ar. The integer “q” indicates the number of R groups on Ar and is 1, 2, 3, 4, or 5.

R₁₀ is independently a hydrogen or a branched or unbranched C₁-C₅ alkyl group. The group Q in formula II is

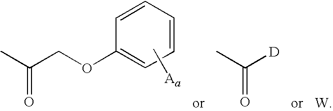

A is independently hydrogen, hydroxyl, halogen, substituted or unsubstituted C₁-C₆ alkyl group, substituted or unsubstituted C₁-C₆ alkoxy group, or substituted or unsubstituted C₄-C₈ alkylene group forming a second ring on Ar. The number of substituents A on the phenyl ring is defined by the integer “a” and is 1, 2, 3, 4, or 5.

D is a substituted or unsubstituted aromatic or heteroaromatic group selected from the group consisting of phenyl, benzyl, phenylamine, toluidinyl, phenacyl, or benzoic acyl.

W is a substituted or unsubstituted aromatic or heteroaromatic group selected from the group consisting of phenyl, pyrimidinyl, tosyl, or pyridinyl.

The invention also relates to the use of compounds of formula III as small molecule inhibitors of HIV:

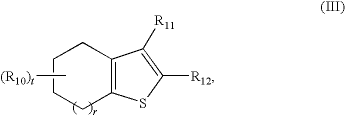

In formula (III), R₁₀ is H, substituted or unsubstituted C₁-C₆ alkyl, substituted or unsubstituted C₄-C₆ cycloalkyl, or substituted or unsubstituted C₁-C₆ alkylene-C₄-C₆ cycloalkyl. Preferably, R₁₀ is H or a branched C₃-C₅ alkyl and more preferably, H or t-butyl.

r is 1 or 2.

t is 0, 1, 2, 3, 4, or 5. Preferably, t is 0 or 1.

R₁₁ is a C(O)₉—(C₁-C₆) alkyl ester and preferably C(O)OC₂H₅.

R₁₂ is a substituted or unsubstituted ring system selected from the group consisting of a C₃-C₁₁ carbocyclic ring system and a C₃-C₁₁ heterocyclic ring system. Preferably, R12 is a heterocyclic ring having one or two ═O and ═S substituents. In a preferred embodiment R₁₂ is the group

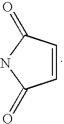

R₁₁ and R₁₂ may also together form a substituted or unsubstituted ring system selected from the group consisting of a C₃-C₁₁ carbocyclic ring system and a C₃-C₁₁ heterocyclic ring system. Preferably, the ring system has one or two ═O and ═S substituents. For example, to form a heterocyclic ring system, R₁₁ and R₁₂ are preferably

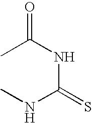

### Pharmaceutical Compositions:

According to the invention, there is also provided a pharmaceutical composition for the treatment of HIV infection, comprising a therapeutically effective amount of a compound of formula I, II or III, a pharmaceutically acceptable salt thereof, or a pharmaceutically effective prodrug thereof and a pharmaceutically acceptable carrier.

As indicated above the small molecule inhibitors of formulas I, II, and III may be used in their neutral form or in the form of a pharmaceutically acceptable salt. “Pharmaceutically acceptable salt” includes those derived from pharmaceutically acceptable acids and bases and which is non-toxic. Examples of pharmacologically acceptable salts include the hydrochloride, hydrobromide, hydroiodide, sulfate, phosphate, acetate, propionate, lactate, maleate, malate, succinate, tartrate salts and the like. All of the pharmacologically acceptable salts may be prepared by conventional means. Na+, K+, and Ca++ salts are also contemplated to be within the scope of the invention. Examples of suitable bases include choline, ethanolamine and ethylenediamine. (See Berge et al, J. Pharm. Sci., 66(1):1-19 (1977) for additional examples of pharmaceutically acceptable salts.)

“Pharmaceutically acceptable prodrugs” represents those prodrugs of the compounds of the present invention which are, within the scope of sound medical judgment, suitable for use in contact with the tissues of humans and lower animals with undue toxicity, irritation allergic response, and the like, commensurate with a reasonable benefit/risk ratio, and effective for their intended use, as well as the zwitterionic forms, where possible, of the compounds of the invention.

“Prodrug” represents compounds which are rapidly transformed in vivo to the parent compound of the above formula, for example, by hydrolysis in blood. A thorough discussion is provided in T. Higuchi & V. Stella, Pro-drugs as Novel Delivery Systems, Vol. 14 of the A. C. S. Symposium Series, and in Edward B. Roche, ed., Bioreversible Carriers in Drug Design, (Am. Pharma. Assoc. and Pergamon Press 1987), both of which are incorporated herein by reference.

“Pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The term “therapeutically effective amount” refers to that amount of a compound of formula that is sufficient to effect treatment, as defined below, when administered to a mammal in need of such treatment. The therapeutically effective amount will vary depending upon the subject and disease condition being treated, the weight and age of the subject, the severity of the disease condition, the manner of administration and the like, which can readily be determined by one of ordinary skill in the art.

Pharmaceutical compositions containing small molecule inhibitors of formulas I, II, or III can be formulated according to known preparatory methods of pharmaceutically useful compositions. A typical pharmaceutical composition includes a pharmaceutically acceptable carrier which may include any solvent, solubilizer, filler, stabilizer, binder, absorbent, base, buffering agent, lubricant, controlled release vehicle, diluent, emulsifying agent, humectant lubricant, dispersion media, coating, antibacterial or antifungal agent, isotonic or absorption delaying agent that is compatible with pharmaceutical administration. The use of such media and agents for pharmaceutically active substances is well-known in the art. Methods for preparing a pharmaceutical composition of an active agent are well known in the art.

A pharmaceutical composition of the invention is formulated to be compatible with its intended route of administration. Examples of routes of administration include parenteral, intravenous, intradermal, subcutaneous, oral, inhalative, transdermal, topical, transmucosal, or rectal administration. Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use may include sterile aqueous solutions (where water soluble) or dispersions and sterile powders for the extemporaneous preparation of sterile injectable solutions or dispersion. For intravenous administration, suitable carriers include, but are not limited to, physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. In the invention, the composition is stable under the conditions of manufacture and storage and can be preserved against the contaminating action of microorganisms such as bacteria and fingi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is desirable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the small molecule inhibitor in the required amount in an appropriate solvent with one or a combination of ingredients enumerated above, as required, followed by filtered sterilization. In many examples, dispersions are prepared by incorporating the active compound into a sterile vehicle which contains a basic dispersion medium and the required other ingredients from those enumerated above. In the case of sterile powders for the preparation of sterile injectable solutions, example methods of preparation include vacuum drying or freeze-drying which yields a powder of the active ingredient plus any additional desired ingredient from a previously sterile-filtered solution thereof.

Oral compositions may include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

Systemic administration may be by transmucosal or transdermal means. For transmucosal or transdermal administration, penetrants appropriate to the barrier to be permeated may be used in the formulation. Such penetrants are generally known in the art, and include, for example, for transmucosal administration, detergents, bile salts, and fusidic acid derivatives. Transmucosal administration may be accomplished through the use of nasal sprays or suppositories. For transdermal administration, a bioactive agent may be formulated into ointments, salves, gels, or creams as generally known in the art.

The therapeutic moieties, which may contain a bioactive compound, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from e.g. Alza Corporation and Nova Pharmaceuticals, Inc. Liposomal suspensions (including liposomes targeted to infected cells with monoclonal antibodies to viral antigens) can also be used as pharmaceutically acceptable carriers.

Oral or parenteral compositions are formulated in dosage unit form for ease of administration and uniformity of dosage. Dosage unit form as used herein includes physically discrete units suited as unitary dosages for the subject to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. In many embodiments, small molecule inhibitors which exhibit large therapeutic indices are selected while small molecule inhibitors that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. In many instances, the dosage of such compounds lies within a range of circulating concentrations that includes the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The invention contemplates a method for the treatment of HIV infection, comprising administering to a patient a therapeutically effective amount of a compound of formula I or II, as described above, alone or in combination with other known antiviral drugs, particularly those used to treat HIV. In such an aspect, the compositions may further comprise other therapeutically effective agents such as chemotherapeutic agents, anti-retroviral inhibitors, cytokines, hydroxyurea, monoclonal antibodies that bind to the Gag proteins, or other inhibitors of retroviral replication. Examples of anti-retroviral inhibitors include nucleoside/nucleotide and non-nucleoside reverse transcriptase inhibitors, protease inhibitors, and fusion inhibitors. In another aspect, using the chemical compounds according to the invention are suitable for therapeutic and prophylactic application in mammals, including humans, suffering from HIV infection.

Also provided by the invention are methods of inhibiting HIV-1 capsid assembly and/or viral replication that comprise the steps of administering into a subject a therapeutic effective amount of the above-mentioned pharmaceutical compositions. In one aspect, the methods further comprise supplementing with an antiviral treatment selected from the group consisting anti-retroviral inhibitors, cytokines, hydroxyurea, monoclonal antibodies that bind to the Gag proteins, or other inhibitors of retroviral replication. Examples of anti-retroviral inhibitors include nucleoside/nucleotide and non-nucleoside reverse transcriptase inhibitors, protease inhibitors, and fusion inhibitors.

Reverse transcriptase inhibitors include nucleoside/nucleotide reverse transcriptase inhibitors (RTIs) and non-nucleoside reverse transcriptase inhibitors (NNRTIs). Nucleoside reverse transcriptase inhibitors, include but are not limited to, Abacavir (ABC; Ziagen), didanosine (dideoxyinosine (ddI); Videx), lamivudine (3TC; Epivir), stavudine (d4T; Zerit, Zerit XR), zalcitabine (dideoxycytidine (ddC); Hivid), zidovudine (ZDV, formerly known as azidothymidine (AZT); Retrovir), abacavir, zidovudine, and lamivudine (Trizivir), zidovudine and lamivudine (Combivir), and emtricitahine (Emtriva). Nucleotide reverse transcriptase inhibitors include tenofovir disoproxil fumarate (Viread). Non-nucleoside reverse transcriptase inhibitors for HIV include, but are not limited to, nevirapine (Virarnune), delavirdine mesylate (Rescriptor), and efavirenz (Sustiva).

Protease inhibitors (PIs) for HIV include amprenavir (Agenerase), saquinavir mesylate (Fortovase, Invirase), ritonavir (Norvir), indinavir sulfate (Crixivan), nelfinavir mesylate (Viracept), lopinavir and ritonavir (Kaletra), atazanavir (Reyataz), and fosamprenavir (Lexiva). Atazanavir and fosamprenavir (Lexiva) are new protease inhibitors that were recently approved by the U.S. Food and Drug Administration (FDA) for treating HIV-1 infection (see Atazanavir (Reyataz) and emtricitabine (Emtriva) for HIV infection (2003), Medical Letter on Drugs and Therapeutics, available online at medletter.com; U.S. Department of Health and Human Services (2003). Guidelines for the Use of Antiretroviral Agents in HIV-Infected Adults and Adolescents. Available online at aidsinfo.nih.gov/guidelines.

Hydroxyurea is a medication used in the treatment of some cancers and sickle-cell anemia. It has been studied for the treatment of HIV infection. Hydroxyurea interferes with the way the HIV makes copies of itself. Studies in the 1990s demonstrated that combinations of hydroxyurea plus one or more nucleoside reverse transcriptase inhibitors (NRTIs) reduced viral load and allowed immune system recovery (measured by increased CD4+ cell counts). In addition, some strains of REV that are normally resistant to certain NRTIs are killed by the combination of hydroxyurea plus the NRTI. Hydroxyurea plus an NRTI and a protease inhibitor (PI) dramatically decreased viral load and increased CD4+ cell counts when given to people in the early stage of HIV infection. The effects of this combination lasted up to seventeen months. Some recent studies, however, have raised concerns about the use of hydroxyurea. One study showed an increased risk of pancreatitis when hydroxyurea was used with didanosine (ddI). Because pancreatitis is a serious health problem, the combination of hydroxyurea and didanosine should be used with caution. Other side effects related to hydroxyurea include a low red blood cell count (anemia) and a low white blood cell count (neutropenia). See U.S. Department of Health and Human Services, 2003, Guidelines for the Use of Antiretroviral Agents in HIV-Infected Adults and Adolescents (available online at aidsinfo.nih.gov and Moore, R. D. et al, AIDS, 15(5):617#20, (2001).

Cytokines are proteins released by cells; examples of cytokines include interferons and interleukins. Cytokines affect the immune system, and they may aid in the production and activation of certain white blood cells (T-lymphocytes) to fight infection. Cytokines also have antiviral and antitumor properties. For example, interferons may be used to treat tumors, including AIDS-related Kaposi's sarcoma (Krensky, A. L. et al., in J. G. Hardman et at, eds., Goodman and Gilman's The Pharmacological Basis of Therapeutics, 10th ed., pp. 1463-1484. New York: McGraw, 2001).

Fusion inhibitors are a new class of drugs recently developed to fight human immunodeficiency virus (HIV). When HIV invades a subject, it attaches to the outside of a CD4+ cell (a type of white blood cell) where it joins (fuses) with the cell and then multiplies. Fusion inhibitors prevent fusion between the virus and the cell from occurring. Therefore, HIV is unable to infect the cell and multiply. An example of a fusion inhibitor of HIV is enfuvirtide (Fuzeon). Lalezari, J. P. et al., New England J. Med., 348:2175-2185, 2003.

### EXAMPLES

### Example 1

### Compound Screening and Capsid Assembly

Compounds. A library of 10,000 drug-like molecules, which a molecular weight is less than or equal to 500 Da, five or fewer H-bond donors, ten or fewer H-bond acceptors, and a calculated log p [octanol/water partition coefficient] less than or equal to 5, was purchased from ChemBridge Corp. (San Diego, Calif.). The average molecular weight was 347 Da (200-596 Da) and was used for subsequent calculations of concentration unless otherwise indicated. The compounds came plated in 96-well plates and solubilized in DMSO at 5 mgs/mL. One or more of the following reagents were used as reference anti-HIV compounds in cell based assays: Indinavir Sulfate, a protease inhibitor was obtained from the NIH AIDS Research and Reference Reagent Program. The following were used as anti-CA HIV compounds: CAP-1 (N-(3-chloro-4-methylphenyl)-N′-{₂-[({5-[(dimethylamino)-methyl]-2-furyl}-methyl)-sulfanyl]-ethyl}urea) compound and DSB (3-O-{3′,3′-dimethylsuccinyl}-betulinic acid).

Soluble HIV-1 capsid protein can be triggered to assemble into tubes similar in diameter and morphology to intact cores by dilution into high iconic strength buffer. The kinetics of assembly can be followed by monitoring the increase in turbidity using a spectrophotometer (Lanham et al., J. Virol., 76:6900, 2002). This assembly assay was adapted to a 96-well, microplate format to allow for medium-throughput screening of compounds by measuring turbidity in a Nepeloskan Ascent plate reader (Thermo Electron Corp., UK). A DIVERSet™ library of 10,000 low molecular weight, “drug-like” compounds was purchased from ChemBridge Corp. (San Diego, Calif.). This library was selected for its potential bioavailability and application based on a pharmacophore diversity analysis. The average molecular weight of the compounds is 347 g/mol, the range being 200-596 g/mol. The compounds came plated in 96-well plates and solubilized in DMSO at 5 mgs/mL.

Turbidity based assay. A 1.5 μL aliquot of compound was diluted 90-fold into 50 mM Na₂HPO₄, 2.52 M NaCl buffer, pH 8 in an optically clear 96-well plate. The plate containing compounds and salt was read to check for compound solubility and to serve as a background reading for the assay. To initiate the assembly reaction, 15 μL of 300 mM stock (in 50 mM Na₂HPO₄ pH 8) of recombinant HIV-1 capsid protein (CA), purified as described (Lanman et al., J. Virol., 76:6900, 2002), was added to each well of the 96-well plate using a multi-channel pipettor. The final average compound concentration was nominally 142 μM, the CA concentration was 30 μM, and the NaCl concentration was 2.25 M. While DMSO (final experimental concentration of 1%) had no effect over the short time scale of the experiment it was necessary to initiate the reactions ⅓ of a plate at a time as extended exposure of unassembled CA to DMSO resulted in decreased assembly. Furthermore, the halftime of the assembly reaction is ˜5 minutes so reading the plate in thirds prevented loss of initial rate data. Therefore, CA was added to the first 4 lanes, the reactions followed for ten minutes, reading was paused, CA was then added to the next four lanes and the process repeated. Instrument settings are as follows: PMT gain of 500 V, lamp at 10V, and integration time of 60 ms. The data were exported to a custom designed Excel spreadsheet that allowed simultaneous plotting of kinetic curves.

About 10% out of 10,000 compounds screened were found to have some inhibitory effect on capsid assembly. The compounds were categorized as either strong, medium, or weak inhibitors based on the approximate degree of inhibition. For the purpose of this invention, “strong” is more than 80%, “medium” is 40%-80% and “weak” is below 40%.

### Example 2

### HTS Assay

Recombinant CA protein was purified as previously described and stored at 300 μM at −80° C. A turbidity based assembly assay, described above, was adapted to a 96-well, microplate format to allow for medium-throughput screening of compounds in a Nepheloskan Ascent plate reader (Thermo Electron Corp., UK). A 1.5 μL aliquot of compound or DMSO was diluted 90-fold into 50 mM Na₂HPO₄, 2.25 M NaCl buffer, pH 8 in an optically clear 96-well plate. To initiate the assembly reaction, 15 μL of the 300 μM stock (in 50 mM Na₂HPO₄ pH 8) of recombinant HIV-1 capsid protein (CA), was added to each well of the 96-well plate using a multi-channel pipettor. The final average compound concentration was nominally 142 μM, the CA concentration was 30 μM, and the NaCl concentration was 2.25 M. The compounds were evaluated for their ability to decrease the rate of assembly as monitored by turbidity and scored by eye into strongly inhibitory, moderately inhibitory, or weakly inhibitory classes.

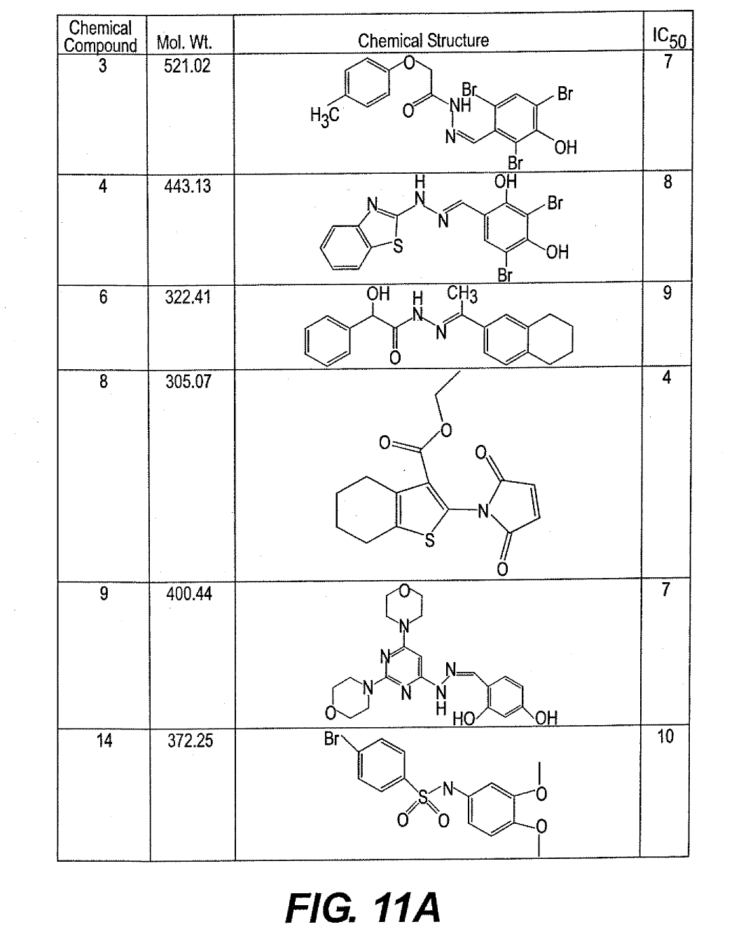

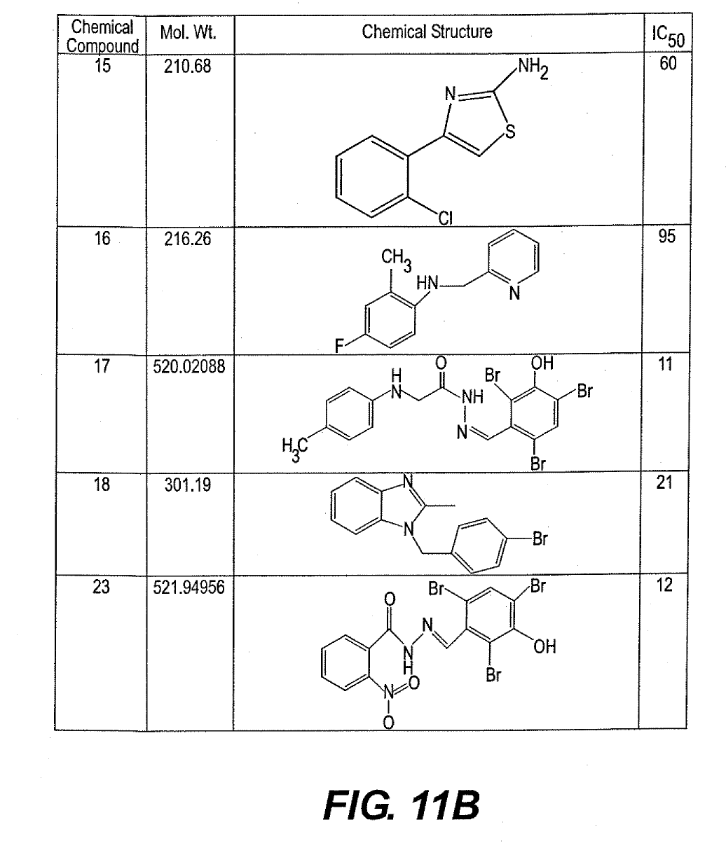

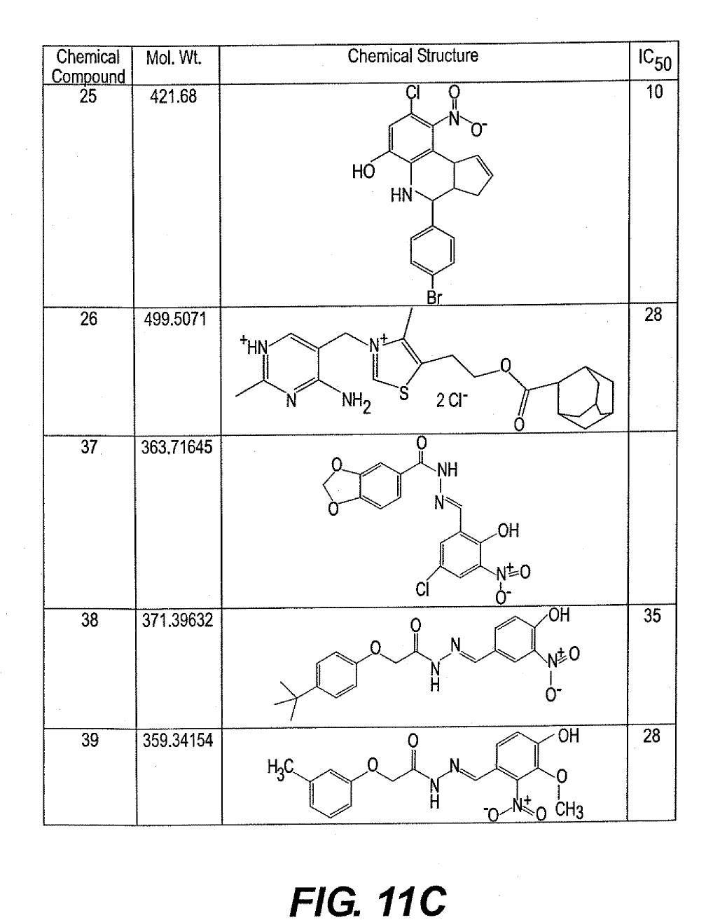

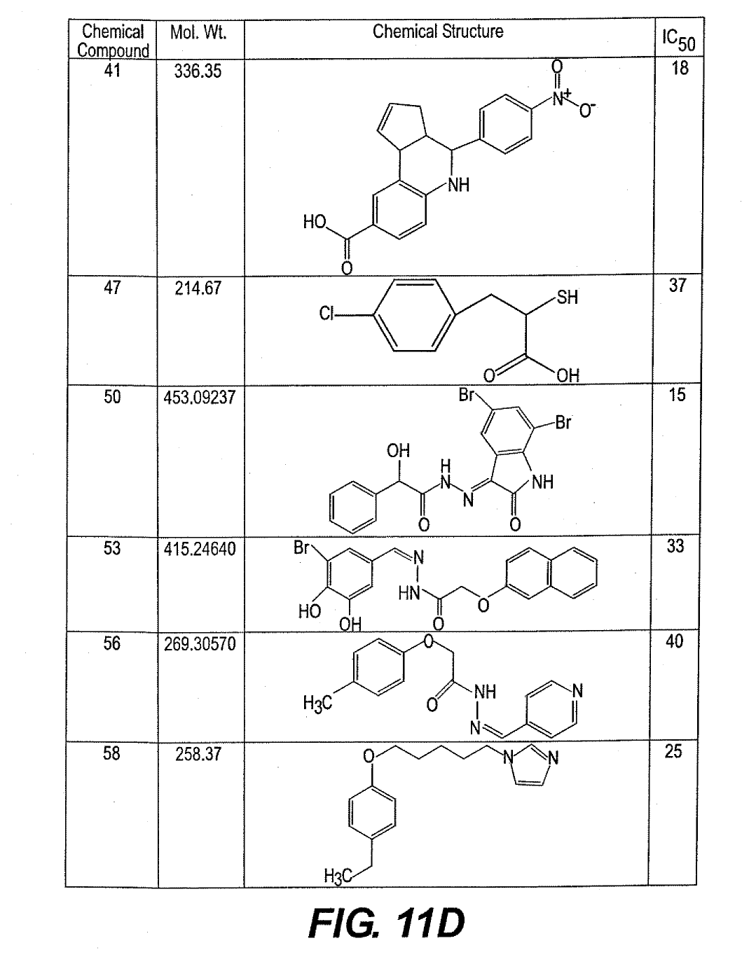

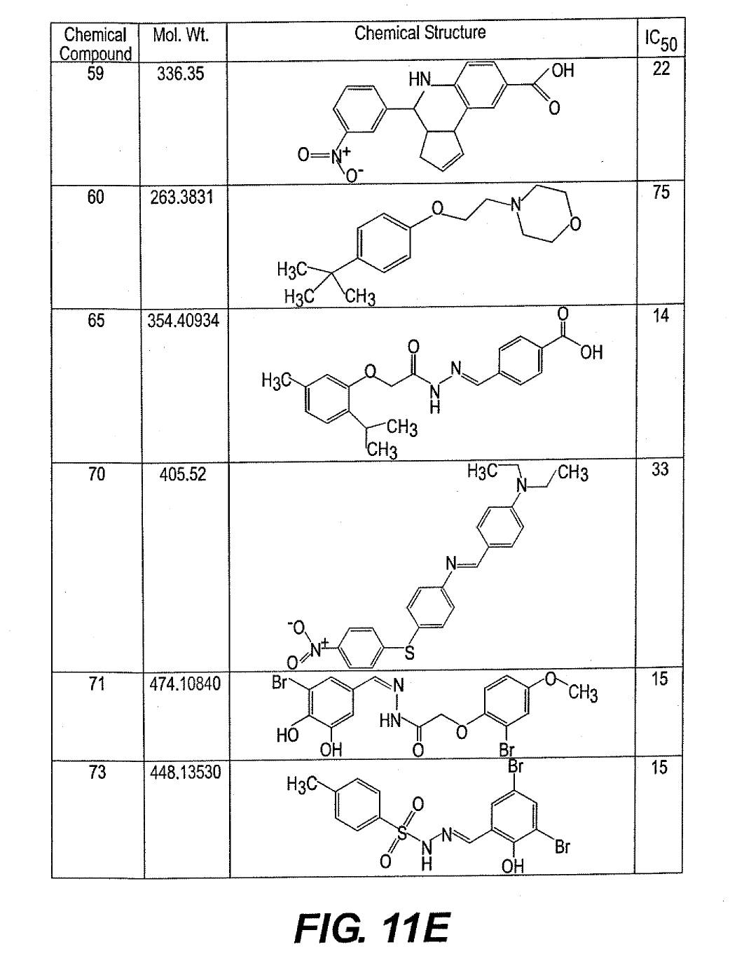

Table 1 summarizes the results of the turbidity assay of the chemical compounds of the invention. FIGS. 11A-E list these chemical compounds, as well as their molecular weights, chemical structures, IC₅₀ data identified according to the invention. The IC₅₀ for the compounds was determined by twofold dilutions of the compounds over a 128 fold range starting from the nominal 142 FM concentration used in the initial screening. The initial assembly rates were determined turbidimetrically and plotted against the compound concentration. The IC₅₀ values ranged from 2 to 100 μM, with an average value of 22 μM. The “Strong Hits” is defined as the IC₅₀ value less than or equal to 40 μM.

TABLE 1
Results of Turbidity Assay of the Compounds

| Compound | MTS | Actual | MTS | Actual | MTS | Actual | MTS AVG 4 | IC₅₀ |
|---|---|---|---|---|---|---|---|---|
| Number | 10 μM | μM | 30 μM | μM | 100 μM | μM | (No Effect) | μM |
| 3 | 4 | 6.72 | 3 | 20.15 | 4 | 66.22 | 3.667 | 7 |
| 4 | 1 | 7.90 | 2 | 23.70 | 1 | 77.86 | 1.333 | 8 |
| 6 | 4 | 10.86 | 3 | 32.57 | 2 | 107.01 | 3.000 | 9 |
| 8 | 4 | 11.46 | 4 | 34.39 | 3 | 112.98 | 3.667 | 4 |
| 9 | 4 | 8.74 | 1 | 26.22 | 1 | 86.16 | 2.000 | 7 |
| 14 | 2 | 7.23 | 1 | 21.70 | 2 | 71.29 | 1.667 | 10 |
| 15 | 4 | 12.78 | 3 | 38.33 | 2 | 125.96 | 3.000 | 60 |
| 16 | 4 | 16.18 | 4 | 48.55 | 1 | 159.53 | 3.000 | 95 |
| 17 | 4 | 4.81 | 1 | 14.42 | 2 | 47.38 | 2.333 | 11 |
| 18 | 1 | 9.68 | 1 | 29.05 | 1 | 95.46 | 1.000 | 21 |
| 23 | 4 | 6.71 | 4 | 20.12 | 3 | 66.10 | 3.667 | 12 |
| 25 | 1 | 8.30 | 1 | 24.90 | 1 | 81.82 | 1.000 | 10 |
| 26 | 4 | 7.01 | 4 | 21.02 | 4 | 69.07 | 4.000 | 28 |
| 37 | 4 | 9.62 | 1 | 28.87 | 1 | 94.85 | 2.000 | |
| 38 | 4 | 9.42 | 3 | 28.27 | 3 | 92.89 | 3.333 | 35 |
| 39 | 4 | 9.74 | 2 | 29.22 | 1 | 96.01 | 2.333 | 28 |
| 41 | 4 | 10.41 | 4 | 31.22 | 3 | 102.57 | 3.667 | 18 |
| 47 | 4 | 16.30 | 1 | 48.91 | 1 | 160.71 | 2.000 | 37 |
| 50 | 4 | 7.72 | 2 | 23.17 | 1 | 76.14 | 2.333 | 15 |
| 53 | 4 | 8.43 | 1 | 25.29 | 1 | 83.08 | 2.000 | 33 |
| 56 | 4 | 13.00 | 3 | 38.99 | 3 | 128.11 | 3.333 | 40 |
| 58 | 2 | 13.55 | 1 | 40.64 | 1 | 133.53 | 1.333 | 25 |
| 59 | 4 | 10.41 | 4 | 31.22 | 4 | 102.57 | 4.000 | 22 |
| 60 | 4 | 13.29 | 4 | 39.87 | 2 | 130.99 | 3.333 | 75 |
| 65 | 4 | 9.88 | 4 | 29.63 | 3 | 97.35 | 3.667 | 14 |
| 70 | | 8.63 | 4 | 25.89 | 2 | 85.08 | 3.000 | 33 |
| 71 | 4 | 7.38 | 2 | 22.15 | 1 | 72.77 | 2.333 | 24 |
| 73 | 4 | 7.81 | 3 | 23.43 | 2 | 76.99 | 3.000 | 15 |

### Example 3

### In Vitro Studies

Cells and Viruses. HEK-293, a human embryonic kidney cell line and TZM-b1, a HeLa based cell line, were maintained in DMEM medium supplemented with 10% (vol/vol) heat-inactivated fetal bovine serum (FBS), 100 U/ml penicillin, 100 μg/ml of streptomycin and 292 μg/ml of L-glutamine. 5.25.GFP.Luc.M7 cells (Luc-M7), a CEM×174 based cell line, were maintained in RPMI 1640 containing 10% (vol/vol) FBS, 100 U/ml penicillin, 100 μg/ml of streptomycin, 292 μg/ml of L-glutamine, 1% Hepes, 0.5 μg/ml puromycin, 0.3 mg/ml geneticin (G418) and 200 μg/ml hygromycin B. Human peripheral blood mononuclear cells (PBMCs) were obtained from healthy donors and isolated by the Ficoll-Hypaque technique. HIV-1_{NL43} and HIV-1_{YU2} were prepared by transient transfection of 293T cells with pNL43 and pYU2, respectively, using FuGene 6 (Roche) and collecting the viral supernatant 48-72 hours post transfection.

Anti-HIV Assays (Multiple Round). The inhibitory effects of the tested compounds on HIV-1_{YU2} replication were determined by the level of luciferase expression after 6 days of infection using Bright-Glo assay (Promega). An amount of 1.5×10⁴ Luc-M7 (a gift from Ned Landau) cells per ml were infected with HIV-1_{YU2} at a multiplicity of infection (MOI) of 3. The HIV-1 infected (0-75 mM compound) or mock-infected (0-300 μM compound) Luc-M7 cells were placed in 96-well culture plates (100 μl/well) with 100 μl of various concentrations of the compounds with 20 μg/ml DEAE Dextran in triplicate and incubated at 37° C. under 5% CO₂ at 100% humidity. After 6 days, cell viability was quantified by Cell-Titer Glo assay (Promega). The 50% toxic concentration (TC₅₀), 50% inhibitory concentration (IC₅₀), and the therapeutic index (TI=TC₅₀/IC₅₀) were then calculated for each of the compounds. The inhibitory effects of the tested compounds on HIV-1_{BAL} and HIV-1_{NL43} viral spread were determined by the level of EGFP expression after 5 days. JLTRG-R5 cells per ml were co-cultured with Molt4 cells/ml stably infected with either HIV-1_{BAL} or HIV-1_{NL43} in 384-well plates in the presence of 0-75 μM compound in singlet. On day 5 cell viability was determined in the mock-infected cells.

Anti-HIV activity was also investigated in PBMCs infected with HIV-1_{YU2} and cultured with various concentrations of test compounds (0-75 μM compound) in triplicate. The activity was evaluated by the level of inhibition of p24 core antigen in the culture supernatant as assessed with the HIV-1 p24 enzyme-linked immunosorbent assay (ELISA) (Beckman-Coulter).

Anti-HIV Assays (Single Round). The inhibitory effect of the compounds on virus production and infectivity were determined by measuring the level of p24 produced in the presence of various concentrations of compounds from HEK-293 cells transiently transfected with pNL43 and by titering the virus on TZM-b1 cells. Briefly, 2.5×10⁵ HEK-293 cells/ml were transfected with pNL43 using FuGene 6. Four hours post transfection the cells were plated in 96-well plates containing 100 μl of various concentrations of compounds (0-75 μM or 0-300 μM for mock transfected cells) in triplicate. At 72-hours post transfection virus supernatant was collected and stored at −80° C. until analysis. The level of p24 produced at each compound concentration was determined relative to the level produced in the presence of the DMSO only controls using HIV-1 p24 enzyme-linked immunosorbent assay (ELISA) (Beckman-Coulter). To determine the infectivity of the virus produced TZM-b1 cells were plated at 1×10⁵ cells/ml in 100 μL in 96-well plates overnight. The next day, the media was replaced with 75 μL DMEM supplemented with 1% FBS, 1× PSG and 40 μg/ml DEAE Dextran and 25 μL of viral supernatant from the HEK-293 cells was added to each well. Three hours later 100 μL of DMEM with 7% FBS was added to each well. Two days after infection the cells were lysed and the level of luciferase expression was determined using the Bright-Glo assay.

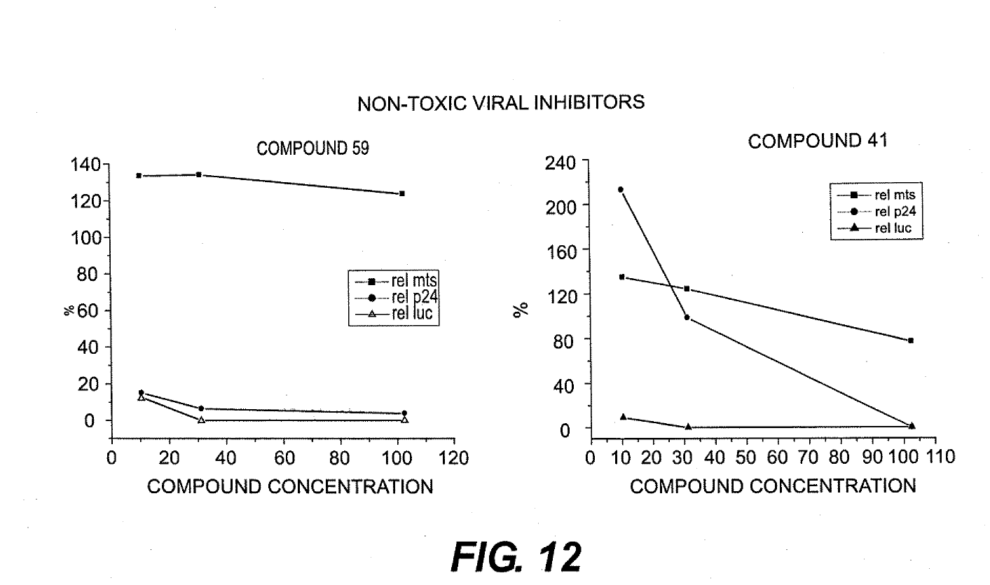

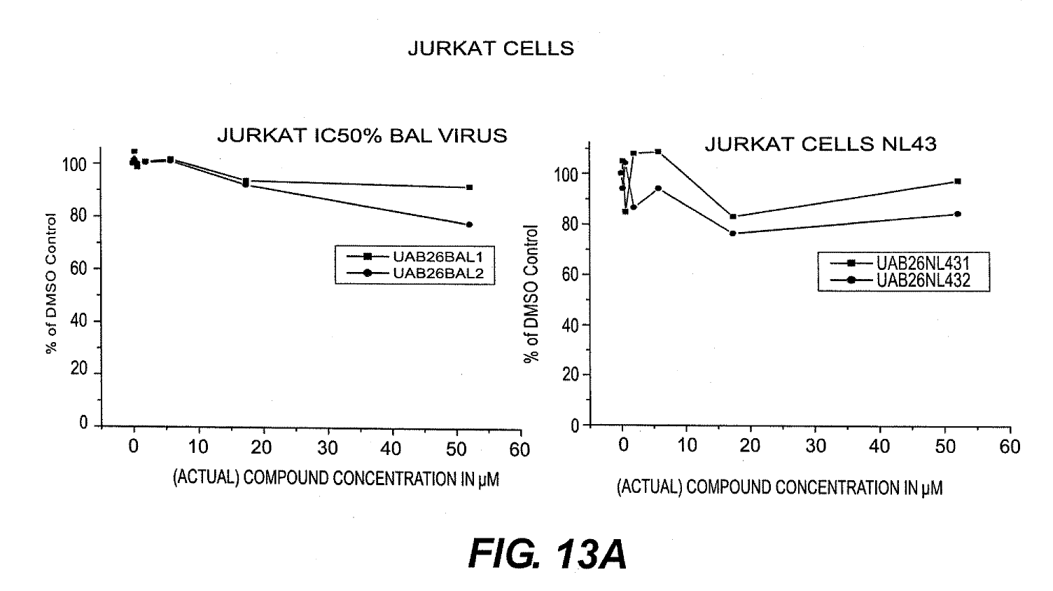

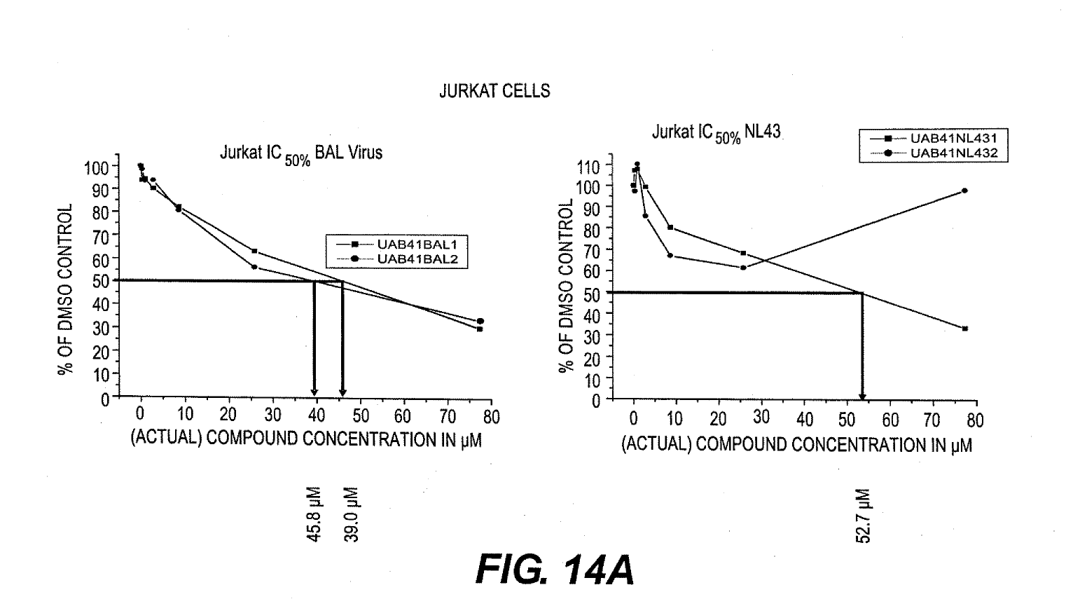

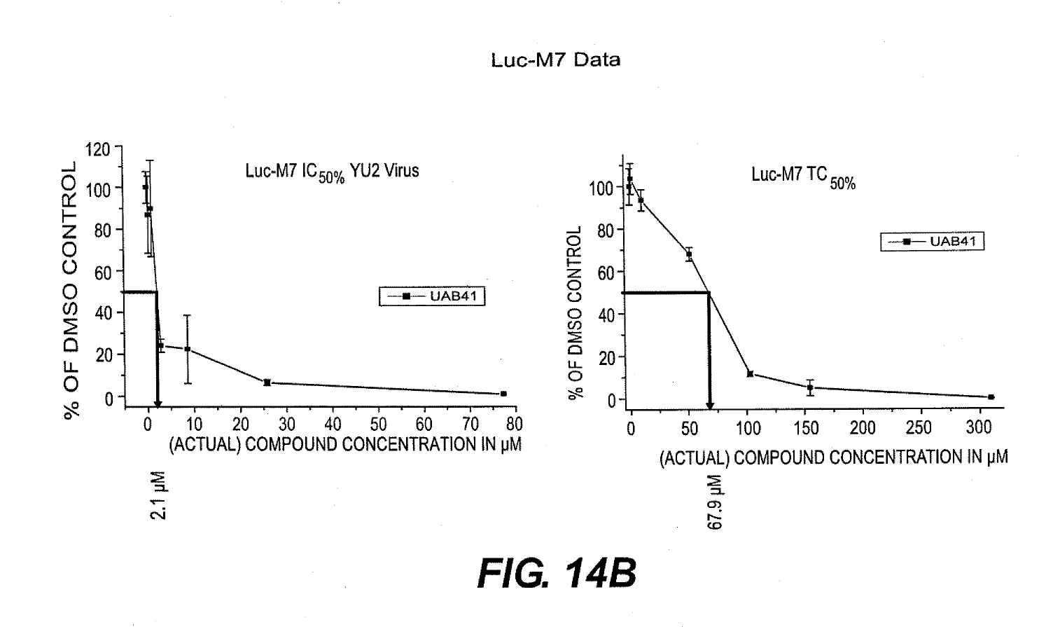

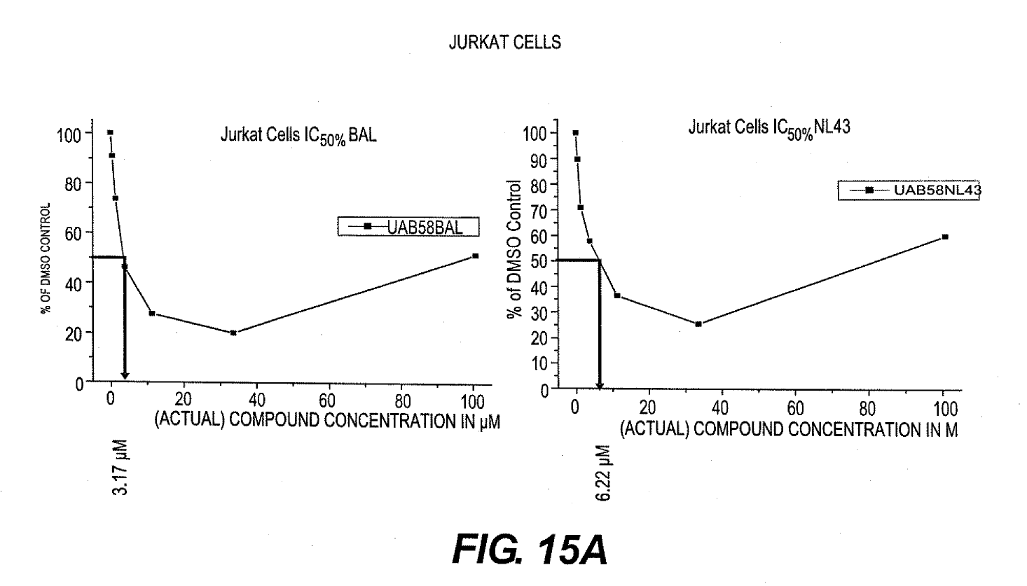

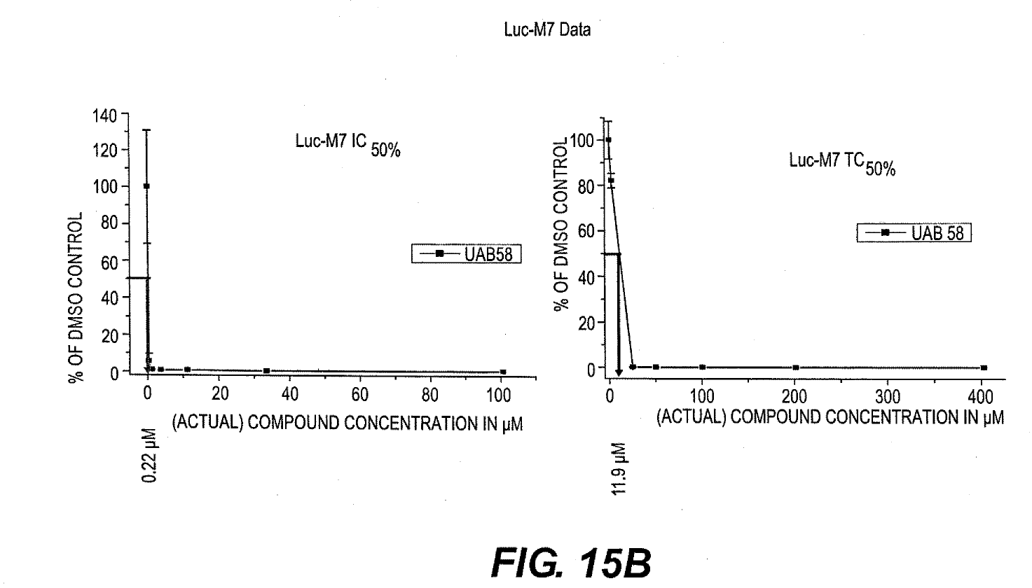

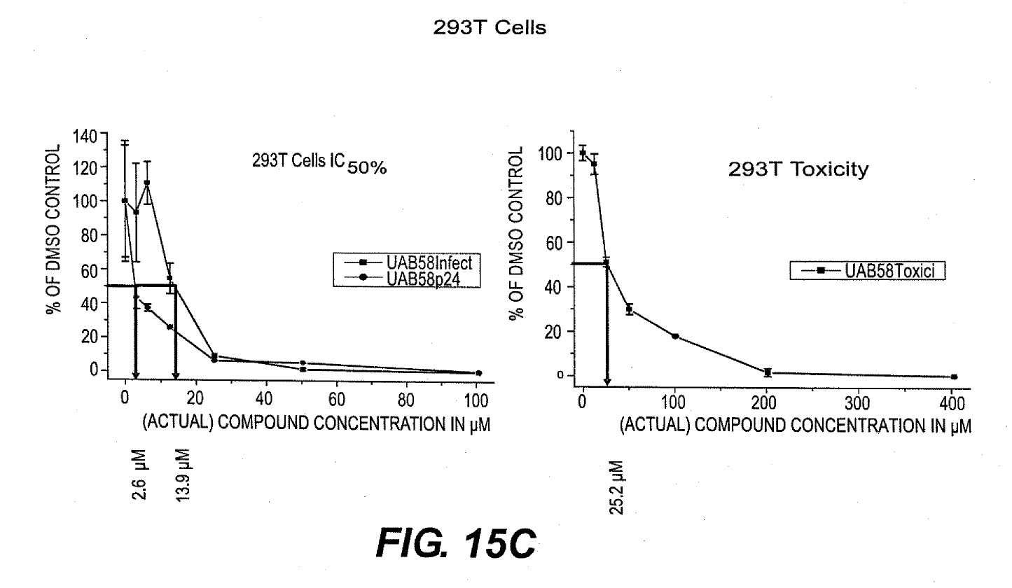

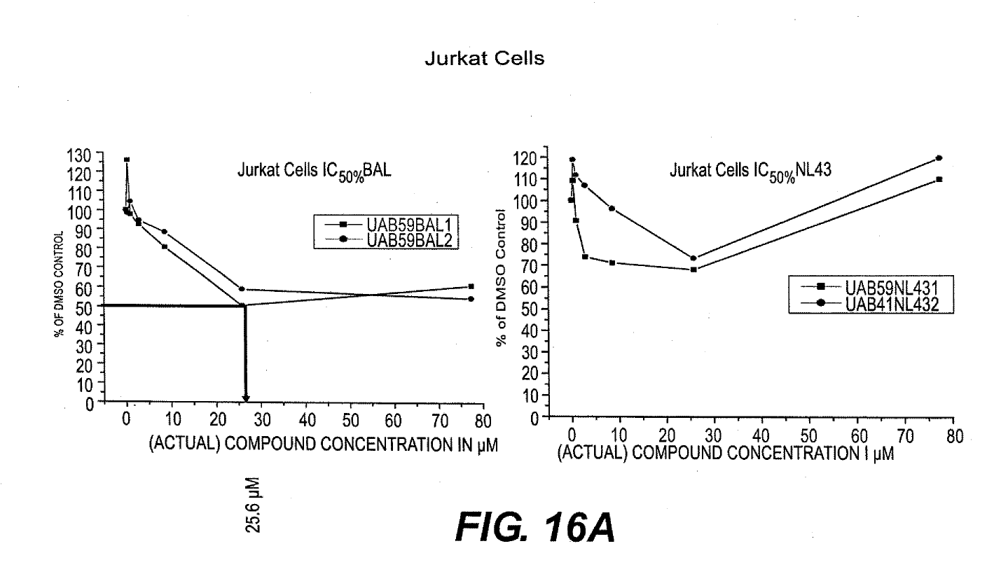

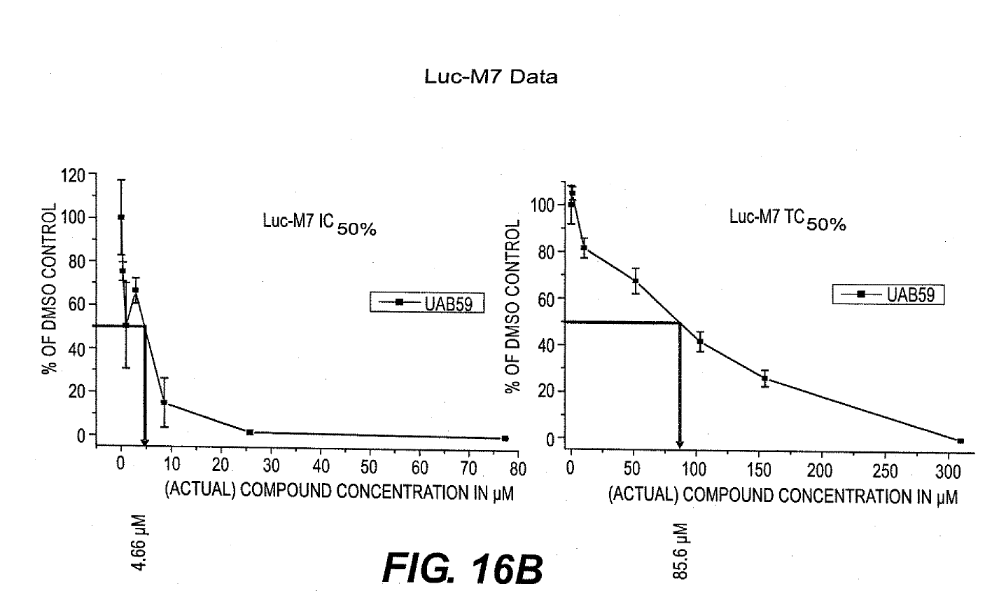

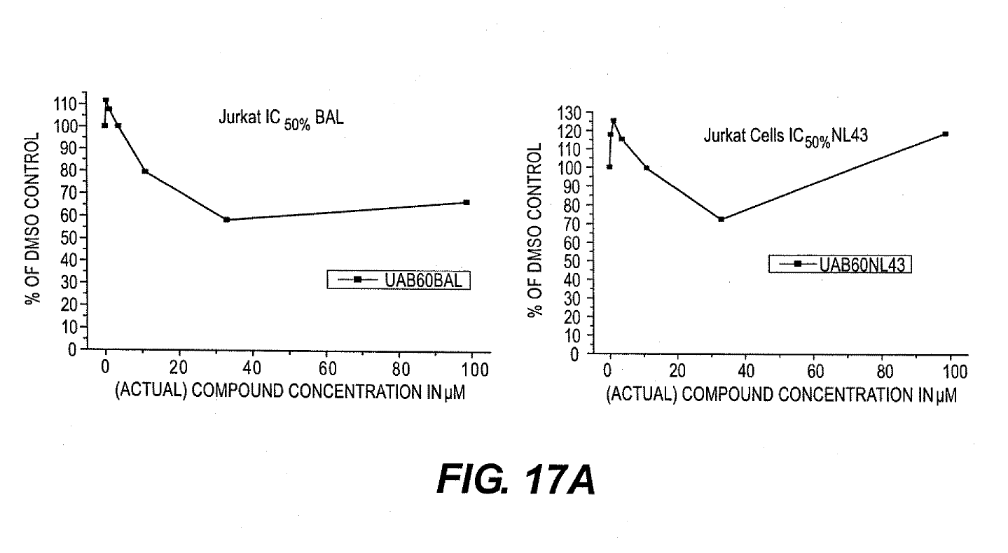

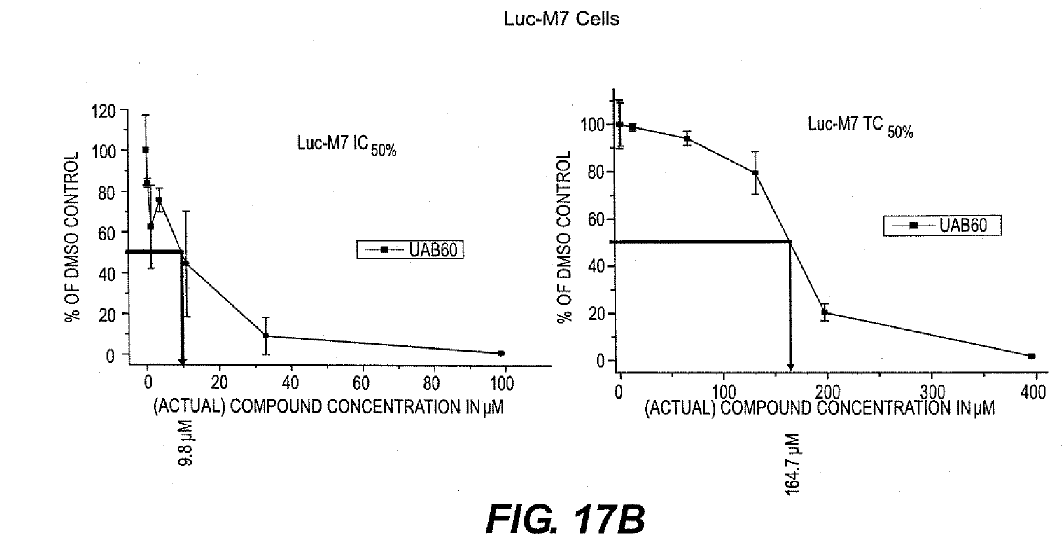

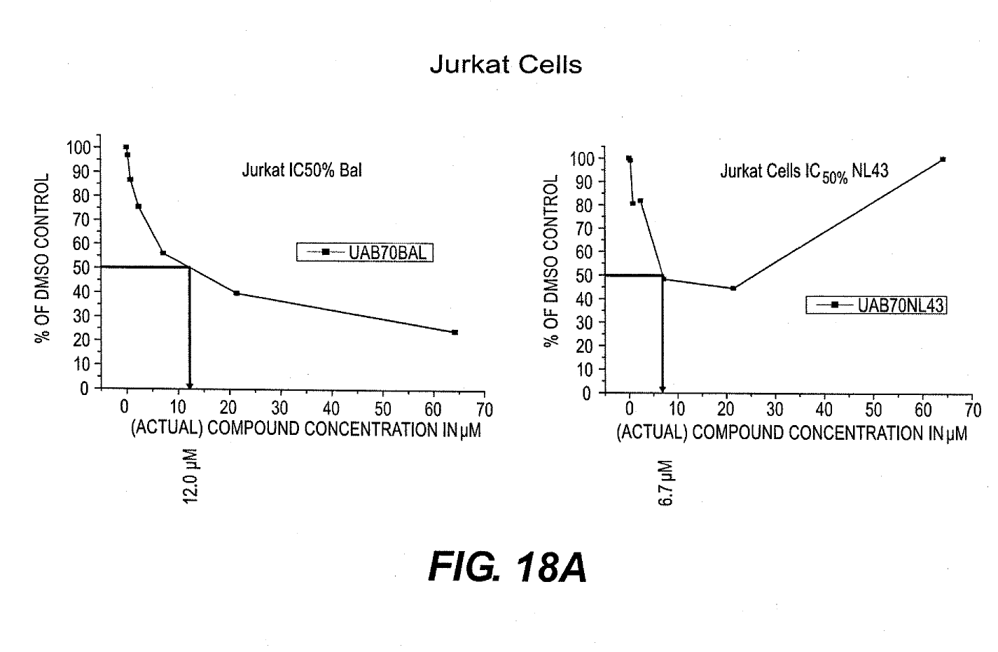

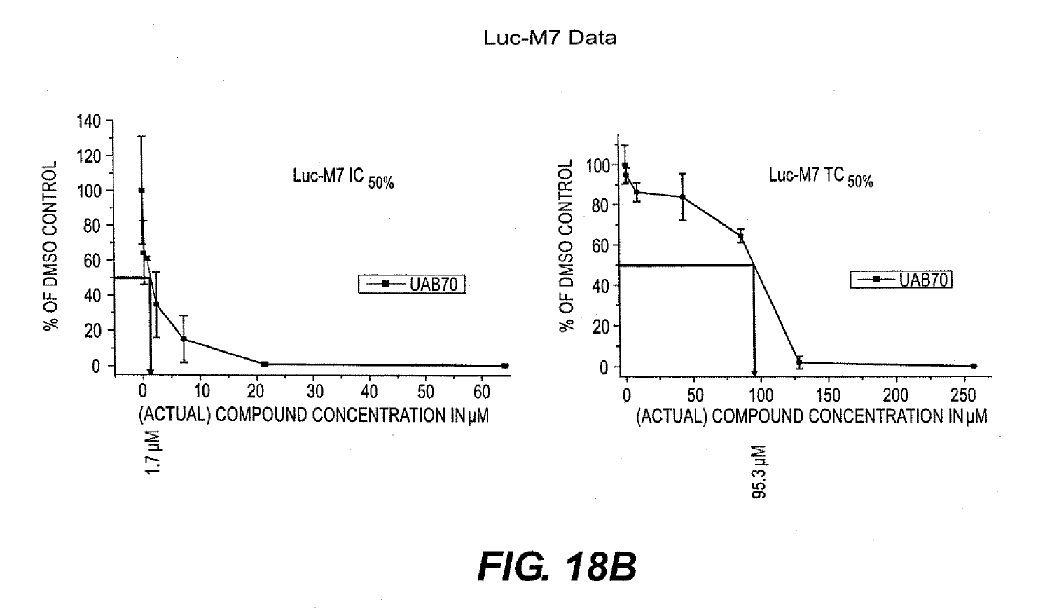

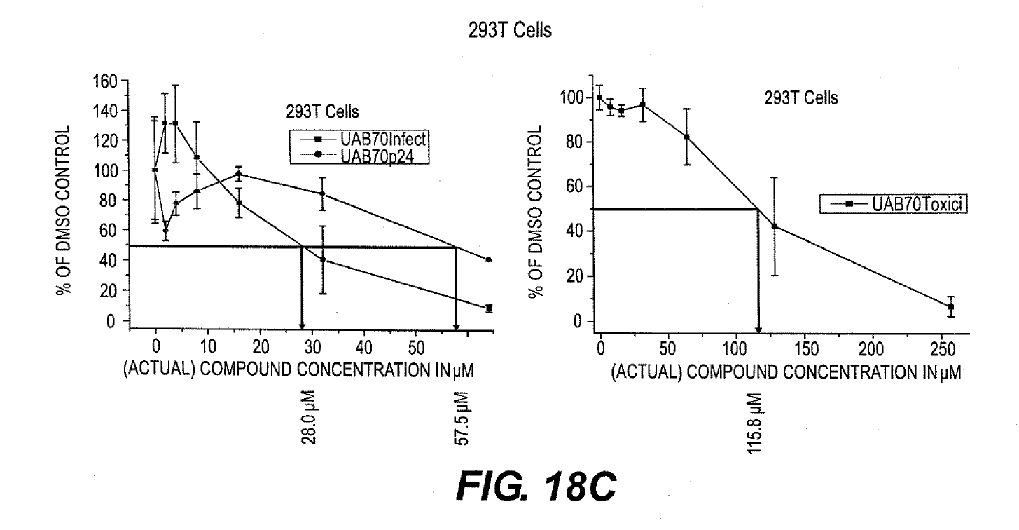

FIGS. 12-18 show the anti-HIV assays for the examples of chemical compounds used according to the invention. After in-vitro assay, the chemical compounds in FIGS. 12-18 were selected for screening for cell viability and toxicity. Jurkat cells assay was used as screening assay. In Jurkat cells assay, JLTRG-R5 cells were used as Indicator cells and MOLT4 cells as Producer cells of NL43 and BAL virus. 96 chemical compounds were plated at seven different concentration from 75 μM to 0 μM at three-fold serial dilutions in 384 well plate. After five days, GFP expression was read. Cell viability was monitored with Alamar Blue assay. Luc M7 assay was designed to measure inhibitory activity of the chemical compounds to viral replication. After infection of cells with YU2 virus, plate cells in 96-well plate in the presence of six concentration of compounds in DMSO. At day 6, the number of cells in wells were read with Cell Titer Glo kit or by counting the number of cells infected with Bright Glo kit. 293T cells assay was designed to determine the toxicity. 293T cells were transfected with NL43. After four hours, transfected cells were plated in 96-well plate in DMSO and 2 μM Indinavir. 72 hours later, viral supernatant was collected and plated on JC53BL. Luciferase expression was measure after two days and p24 analysis was conducted with 1:1000 and 1:2000 dilutions.

TI (therapeutic Index) table in Luc-M7 cells for the examples of the small molecule inhibitor compounds were summarized in Table 2 and TI in 293T cells were summarized in Table 3.

TABLE 2
Therapeutic Index Table in Luc-M7 Cells

| Compound | IC50 | TC50 | TI |
|---|---|---|---|
| 58 | 0.06 | 8.7 | 145.00 |
| 70 | 1.47 | 92.5 | 62.93 |
| 41 | 2.1 | 65.8 | 31.33 |
| 59 | 4.5 | 84.1 | 18.69 |
| 60 | 6.98 | 125.3 | 17.95 |
| Achillion | 4.11 | 64.8 | 15.77 |
| 26 | 4.57 | 70.4 | 15.40 |
| 16 | 17.8 | 66.5 | 3.74 |
| 15 | 19.79 | 58.9 | 2.98 |
| 6 | 15.4 | 36.8 | 2.39 |
| 33 | 36.8 | 77.2 | 2.10 |
| 8 | 13.3 | 27.7 | 2.08 |

IC₅₀: 50% Inhibitory Concentration
TC₅₀: 50% toxicity Concentration
TI: Therapeutic Index (Toxicity/Efficacy: TC₅₀/IC₅₀)
Active: TI > 10
Moderate: <1.5 TI < 10
Inactive: TI < 1.5

TABLE 3
Therapeutic Index in 293T cells

| | Infectivity | | | TI - | |
|---|---|---|---|---|---|
| Compound | 50% | p24 50% | TC50% | infectivity | TI - p24 |
| 26 | 17.6 | 60.2 | 193 | 10.97 | 3.21 |
| 73 | 32 | 70 | 178 | 5.58 | 2.55 |
| 47 | 34.2 | 24.6 | 202 | 5.91 | 8.21 |
| 25 | 17.7 | 10.4 | 174 | 9.83 | 16.7 |
| 14 | >75 | 62.8 | 102 | N/R | 1.62 |
| 70 | 32.9 | 67.5 | 135 | 4.11 | 2.00 |
| 58 | 10 | 1.69 | 5 | 0.50 | 2.96 |
| 18 | 43.2 | 1.8 | 9.1 | 0.21 | 5.06 |

IC₅₀: 50% Inhibitory Concentration
TC₅₀: 50% toxicity Concentration
TI: Therapeutic Index (Toxicity/Efficacy: TC₅₀/IC₅₀)
Active: TI > 10
Moderate: <1.5 TI < 10
Inactive: TI < 1.5

Compound 26 has excellent TI value in both Luc-M7 cells and 293T cells. Saturation Transfer Difference NMR (STD-NMR) experiment was conducted with the compound 26 to confirm the binding with CA protein. The STD-NMR spectroscopy is an extremely powerful and sensitive technique to detect and identify the binding of low molecular weight (MW) ligands specific to a target protein of interest. It is particularly useful in identifying potential lead compounds that bind target proteins of interest typically only with a weak affinity (K_{d} in mM to μM range). This technique exploits the transfer of radio frequency saturation of the proton magnetization on the large protein to the low MW ligand during the brief period it resides within the binding pocket of the protein, before it is released into solution. It also exploits the large differences in the correlation times of the interacting molecules. This saturation transfer serves to label the ligand that is recognized by the protein (i.e., ligands that do not bind the protein are not labeled by saturation transfer).

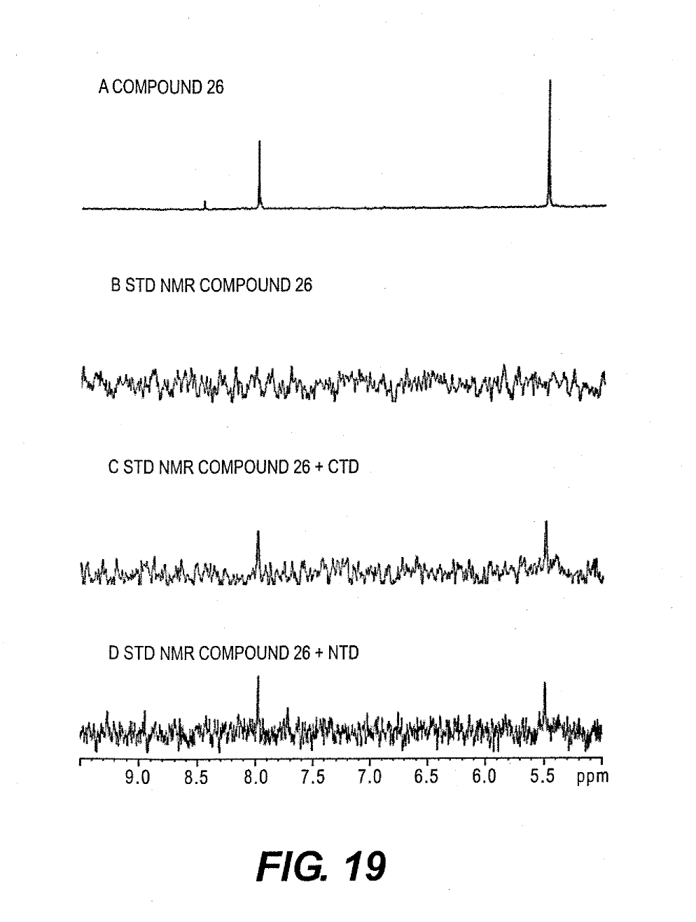

FIGS. 19A-D show the STD-NMR spectra of the compound 26. FIG. 19A represents the reference ID-NMR spectrum of the low-field region of a solution of compound 26 by itself; at 1 mM concentration, recorded on a Bruker 600 MHz NMR spectrometer. The two large peaks (at 5.5 ppm and 8 ppm) are from the CH protons attached to the two rings in the structure. FIG. 19B shows the STD-NMR spectrum of this compound by itself (obtained by irradiating at −1 ppm for on-resonance condition). It may be noted that the STD-N spectrum shows no peaks, as expected. FIG. 19C shows the STD-NMR spectrum obtained under identical conditions, except a trace amount (0.02 mM) of CUD was added to the sample used in FIG. 19A. The binding of the chemical compound 26 to the CTD domain is clearly indicated by the two peaks at 5.5 ppm and 8 ppm. FIG. 19D shows the STD-NMR spectrum obtained under identical conditions, except the NTD domain was added to the solution containing the compound 26. Once again, both the peaks at 5.5 ppm and 8 ppm light up in this spectrum, indicating that compound 26 binds to the NTD domain as well. Thus, these data unequivocally establish that compound 26 is binding to both the NTD and CTD domains separately. This method can be used to identify additional compounds recognized by both domains of CA separately, and by the full-length CA protein.

Any patents or publications mentioned in this specification are indicative of the levels of those skilled in the art to which the invention pertains. These patents and publications are herein incorporated by reference to the same extent as if each individual publication was specifically incorporated by reference.

One skilled in the art will readily appreciate that the invention is well adapted to carry out the objects and obtain the ends and advantages mentioned, as well as those inherent therein. The examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

The claimed invention is:

1. A pharmaceutical composition for the treatment of an HIV infection comprising an effective amount of a compound of formula I, a pharmaceutically acceptable salt thereof, or a pharmaceutically prodrug thereof, and a pharmaceutically acceptable carrier 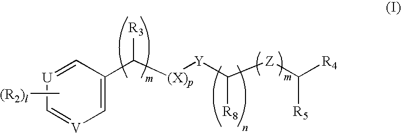
wherein:
U is CR₁ or N;
V is CR₁ or N;
R₁ and R₂ are independently hydrogen, hydroxyl, halogen, carboxyl, nitro, —NH₂, —NHR₆, —NR₆R₇, substituted or unsubstituted C₁-C₆ alkyl, substituted or unsubstituted C₂-C₆ alkenyl, and substituted or unsubstituted C₂-C₆ alkynyl, substituted or unsubstituted C₁-C₆ alkoxyl group, C₃-C₇ alkylene forming a substituted or unsubstituted second ring on the ring containing U and V, or C₁-C₄ amide group forming a substituted or unsubstituted second ring on phenyl ring, an aliphatic or aromatic ring substituent selected from the group consisting of substituted or unsubstituted C₃-C₈ cycloalkyl, substituted or unsubstituted C₅-C₈ cycloalkenyl and aryl wherein said aliphatic or aromatic ring substituent may be substituted with one or more 5- or 6-membered aromatic or aliphatic heterocyclic groups;
l is 1, 2, or 3;
R₆ and R₇ are independently substituted or unsubstituted C₁-C₆ alkyl, substituted or unsubstituted C₄-C₆ cycloalkyl, or substituted or unsubstituted C₁-C₆ alkylene-C₄-C₆ cycloalkyl;
R₃ is hydrogen, hydroxyl, substituted or unsubstituted C₁-C₆ alkyl, a bond which forms a double bond with X when X is a nitrogen, together with Y and R₁ or R₂ form a substituted or unsubstituted 5- to 11-membered heterocyclic ring; or together with Y and R₈ form a substituted or unsubstituted 5- to 11-membered heterocyclic ring;
n is 0, 1, 2, 3, 4, 5, 6, 7, or 8;
m is 0 or 1;
p is 0 or 1;
X is SO₂, C═O, N, or NH;
Y is NH, N, O, S, NC═O, or substituted or unsubstituted phenylene;
Z is O, SO₂, C═O, NH, or S(O)₂NH;
R₄ and R₅ are independently H, OH, SH, carboxyl, or together form a substituted or unsubstituted ring system selected from the group consisting of a C₃-C₁₁ carbocyclic ring system, a C₃-C₁₁ heterocyclic ring system, aryl, and heteroaryl; and
R₈ is H, OH, SH, carboxyl, substituted or unsubstituted C₁-C₆ alkyl, or a substituted or unsubstituted ring system selected from the group consisting of a C₃-C₁₁, carbocyclic ring system, a C₃-C₁₁ heterocyclic ring system, aryl, and heteroaryl.

2. A pharmaceutical composition of claim 1, wherein U and V are CR₁.

3. A pharmaceutical composition of claim 1, wherein U and V are N.

4. A pharmaceutical composition of claim 1, wherein Y is S; l is 1, 2, or 3; and n is the integer 0, 1, or 2.

5. A pharmaceutical composition of claim 1, wherein Y is NH.

6. A pharmaceutical composition of claim 1, wherein Y is N.

7. A pharmaceutical composition of claim 1, wherein Y is O.

8. A pharmaceutical composition of claim 1, wherein the compound of formula I is

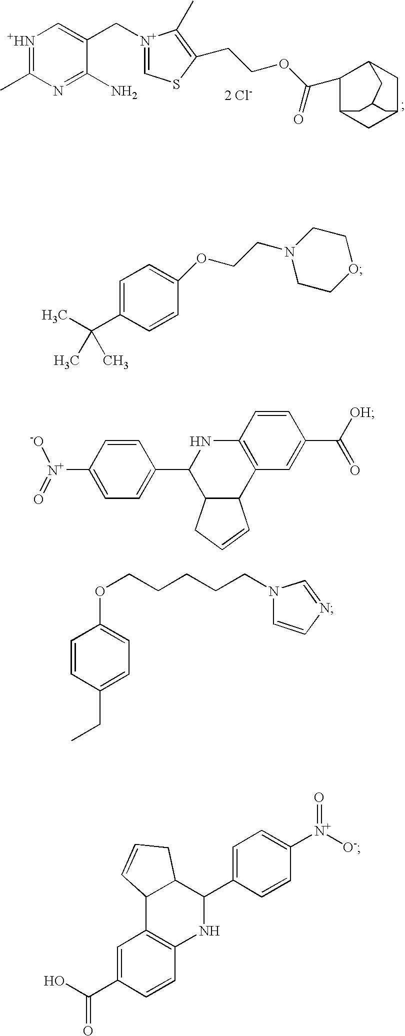

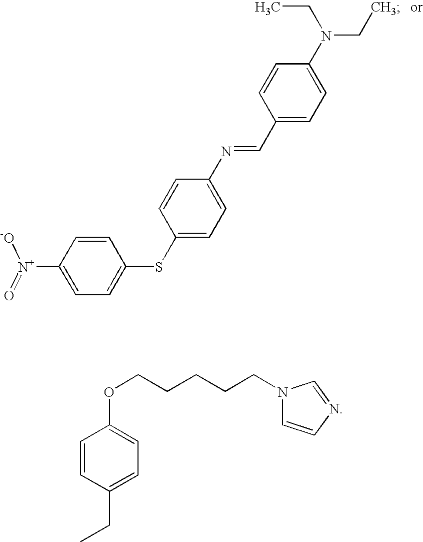

9. A method for inhibiting HIV replication in cells comprising administering to said cells a composition comprising a compound of formula I, a pharmaceutically acceptable salt thereof, or a pharmaceutically prodrug thereof, and a pharmaceutically acceptable carrier 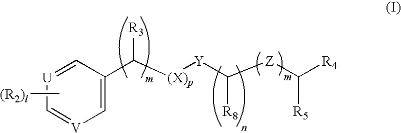
wherein:
U is CR₁ or N;
V is CR₁, or N;
R₁ and R₂ are independently hydrogen, hydroxyl, halogen, carboxyl, nitro, —NH₂, —NHR₆, —NR₆R₇, substituted or unsubstituted C₁-C₆ alkyl, substituted or unsubstituted C₂-C₆ alkenyl, and substituted or unsubstituted C₂-C₆ alkynyl, substituted or unsubstituted C₁-C₆ alkoxyl group, C₃-C₇ alkylene forming a substituted or unsubstituted second ring on the ring containing U and V, or C₁-C₄ amide group forming a substituted or unsubstituted second ring on phenyl ring, an aliphatic or aromatic ring substituent selected from the group consisting of substituted or unsubstituted C₃-C₈ cycloalkyl, substituted or unsubstituted C₅-C₈ cycloalkenyl and aryl wherein said aliphatic or aromatic ring substituent may be substituted with one or more 5- or 6-membered aromatic or aliphatic heterocyclic groups;
l is 1, 2, or 3;
R₆ and R₇ are independently substituted or unsubstituted C₁-C₆ alkyl, substituted or unsubstituted C₄-C₆ cycloalkyl, or substituted or unsubstituted C₁-C₆ alkylene-C₄-C₆ cycloalkyl;
R₃ is hydrogen, hydroxyl, substituted or unsubstituted C₁-C₆ alkyl, a bond which forms a double bond with X when X is a nitrogen, together with Y and R₁ or R₂ form a substituted or unsubstituted 5- to 11-membered heterocyclic ring; or together with Y and R₈ form a substituted or unsubstituted 5- to 11-membered heterocyclic ring;
n is 0, 1, 2, 3, 4, 5, 6, 7, or 8;
m is 0 or 1;
p is 0 or 1;
X is SO₂, C═O, N, or NH;
Y is NH, N, O, S, NC═O, or substituted or unsubstituted phenylene;
Z is O, SO₂, C═O, NH, or S(O)₂NH;
R₄ and R₅ are independently H, OH, SH, carboxyl, or together form a substituted or unsubstituted ring system selected from the group consisting of a C₃-C₁₁ carbocyclic ring system, a C₃-C₁₁ heterocyclic ring system, aryl, or heteroaryl; and R₈ is H, OH, SH, carboxyl, substituted or unsubstituted C₁-C₆ alkyl, or a substituted or unsubstituted ring system selected from the group consisting of a C₃-C₁₁ carbocyclic ring system, a C₃-C₁₁ heterocyclic ring system, aryl, or heteroaryl.

10. The method of claim 9, wherein the composition further comprises a therapeutically effective agent selected from the group consisting of chemotherapeutic agents, anti-retroviral inhibitors, cytokines, hydroxyurea, monoclonal antibodies that bind to the GAG proteins, and combinations thereof.

11. The method of claim 10, wherein the anti-retroviral inhibitors are selected from the group consisting of nucleoside reverse transcriptase inhibitors, nucleotide reverse transcriptase inhibitors, non-nucleoside reverse transcriptase inhibitors, protease inhibitors, and fusion inhibitors.

12. The method of claim 9, wherein the compound of formula I is selected from the group of compounds listed in Table 2.

13. Use of a compound of formula I to treat an HIV infection, comprising the step of administering to a human in need of such treatment a therapeutically effective amount of a compound of formula I 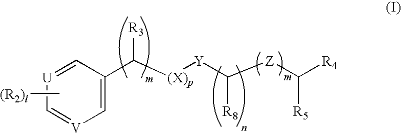
wherein:
U is CR₁ or N;
V is CR₁ or N;
R₁ and R₂ are independently hydrogen, hydroxyl, halogen, carboxyl, nitro, —NH₂, —NHR₆, —NR₆R₇, substituted or unsubstituted C₁-C₆ alkyl, substituted or unsubstituted C₂-C₆ alkenyl, and substituted or unsubstituted C₂-C₆ alkynyl, substituted or unsubstituted C₁-C₆ alkoxyl group, C₃-C₇ alkylene forming a substituted or unsubstituted second ring on the ring containing U and V, or C₁-C₄ amide group forming a substituted or unsubstituted second ring on phenyl ring, an aliphatic or aromatic ring substituent selected from the group consisting of substituted or unsubstituted C₃-C₈ cycloalkyl, substituted or unsubstituted C₅-C₈ cycloalkenyl and aryl wherein said aliphatic or aromatic ring substituent may be substituted with one or more 5- or 6-membered aromatic or aliphatic heterocyclic groups;
l is 1, 2, or 3;
R₆ and R₇ are independently substituted or unsubstituted C₁-C₆ alkyl, substituted or unsubstituted C₄-C₆ cycloalkyl, or substituted or unsubstituted C₁-C₆ alkylene-C₄-C₆ cycloalkyl;
R₃ is hydrogen, hydroxyl, substituted or unsubstituted C₁-C₆ alkyl, a bond which forms a double bond with X when X is a nitrogen, together with Y and R₁ or R₂ form a substituted or unsubstituted 5- to 11-membered heterocyclic ring; or together with Y and R₈ form a substituted or unsubstituted 5- to 11-membered heterocyclic ring;
n is 0, 1, 2, 3, 4, 5, 6, 7, or 8;
m is 0 or 1;
p is 0 or 1;
X is SO₂, C═O, N, or NH;
Y is NH, N, O, S, NC═O, or substituted or unsubstituted phenylene;
Z is O, SO₂, C═O, NH, or S(O)₂NH;
R₄ and R₅ are independently H, OH, SH, carboxyl, or together form a substituted or unsubstituted ring system selected from the group consisting of a C₃-C₁₁ carbocyclic ring system, a C₃-C₁₁ heterocyclic ring system, aryl, and heteroaryl; and
R₈ is H, OH, SH, carboxyl, substituted or unsubstituted C₁-C₆ alkyl, or a substituted or unsubstituted ring system selected from the group consisting of a C₃-C₁₁ carbocyclic ring system, a C₃-C₁₁ heterocyclic ring system, aryl, and heteroaryl.

14. A pharmaceutical composition for the treatment of an HIV infection comprising an effective amount of a compound of formula II, a pharmaceutically acceptable salt thereof, or a pharmaceutically prodrug thereof, and a pharmaceutically acceptable carrier 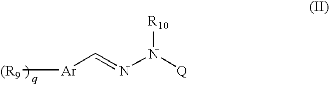
wherein:
Ar is a phenyl group or a pyridinyl group, which bear the substituent R₉;
R₉ is independently a hydrogen, hydroxyl group, halogen, carboxyl, nitro oxide, C₁-C₆ alkoxyl group, C₃-C₇ alkylene group forming a second ring on Ar, or C₁-C₄ amide group forming a second ring on Ar.
q is 1, 2, 3, 4, or 5;
R₁₀ is independently a hydrogen or a branched or unbranched C₁-C₅ alkyl group;
Q is 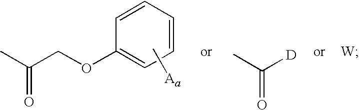
A is independently hydrogen, hydroxyl, halogen, substituted or unsubstituted C₁-C₆ alkyl group, substituted or unsubstituted C₁-C₆ alkoxy group, or substituted or unsubstituted C₄-C₈ alkylene group forming a second ring on Ar;
a is 1, 2, 3, 4, or 5;
D is a substituted or unsubstituted aromatic or heteroaromatic group selected from the group consisting of phenyl, benzyl, phenylamine, toluidinyl, phenacyl, and benzoic acyl; and
W is a substituted or unsubstituted aromatic or heteroaromatic group selected from the group consisting of phenyl, pyrimidinyl, tosyl, and pyridiny.

15. The pharmaceutical composition of claim 14, wherein R₉—Ar of formula II is

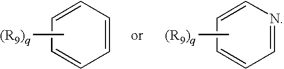

16. A method for inhibiting HIV replication in cells comprising administering to said cells a composition comprising a compound of formula II, a pharmaceutically acceptable salt thereof, or a pharmaceutically prodrug thereof and a pharmaceutically acceptable carrier 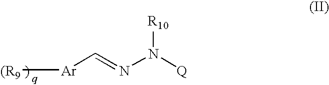
wherein:
Ar is a phenyl group or a pyridinyl group, which bear the substituent R₉;
R₉ is independently a hydrogen, hydroxyl group, halogen, carboxyl, nitro oxide, C₁-C₆ alkoxyl group, C₃-C₇ alkylene group forming a second ring on Ar, or C₁-C₄ amide group forming a second ring on Ar.
q is 1, 2, 3, 4, or 5;
R₁₀ is independently a hydrogen or a branched or unbranched C₁-C₅ alkyl group;
Q is 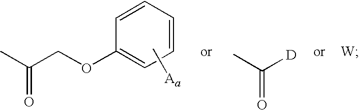
A is independently hydrogen, hydroxyl, halogen, substituted or unsubstituted C₁-C₆ alkyl group, substituted or unsubstituted C₁-C₆ alkoxy group, or substituted or unsubstituted C₄-C₈ alkylene group forming a second ring on Ar;
a is 1, 2, 3, 4, or 5;
D is a substituted or unsubstituted aromatic or heteroaromatic group selected from the group consisting of phenyl, benzyl, phenylamine, toluidinyl, phenacyl, and benzoic acyl; and
W is a substituted or unsubstituted aromatic or heteroaromatic group selected from the group consisting of phenyl, pyrimidinyl, tosyl, and pyridinyl.

17. Use of a compound of formula II to treat an HIV infection comprising the step of administering to a human in need of such treatment a therapeutically effective amount of a compound of formula II 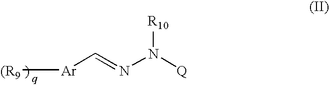
wherein:
Ar is a phenyl group or a pyridinyl group, which bear the substituent R₉;
R₉ is independently a hydrogen, hydroxyl group, halogen, carboxyl, nitro oxide, C₁-C₆ alkoxyl group, C₃-C₇ alkylene group forming a second ring on Ar, or C₁-C₄ amide group forming a second ring on Ar.
q is 1, 2, 3, 4, or 5;
R₁₀ is independently a hydrogen or a branched or unbranched C₁-C₅ alkyl group;
Q is 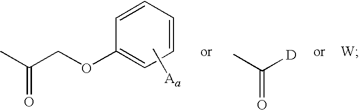
A is independently hydrogen, hydroxyl, halogen, substituted or unsubstituted C₁-C₆ alkyl group, substituted or unsubstituted C₁-C₆ alkoxy group, or substituted or unsubstituted C₄-C₈ alkylene group forming a second ring on Ar;
a is 1, 2, 3, 4, or 5;
D is a substituted or unsubstituted aromatic or heteroaromatic group selected from the group consisting of phenyl, benzyl, phenylamine, toluidinyl, phenacyl, and benzoic acyl; and
W is a substituted or unsubstituted aromatic or heteroaromatic group selected from the group consisting of phenyl, pyrimidinyl, tosyl, and pyridinyl.

18. A pharmaceutical composition for the treatment of an HIV infection comprising an effective amount of a compound of formula III, a pharmaceutically acceptable salt thereof; or a pharmaceutically prodrug thereof, and a pharmaceutically acceptable carrier 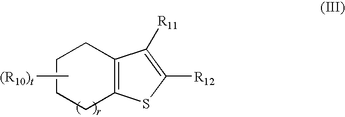
wherein;
R₁₀ is H, substituted or unsubstituted C₁-C₆ alkyl, substituted or unsubstituted C₄-C₆ cycloalkyl, or substituted or unsubstituted C₁-C₆ alkylene-C₄-C₆ cycloalkyl;
r is 1 or 2;
t is 0, 1, 2, 3, 4, or 5; and
R₁₁ is a C(O)O—(C₁-C₆) alkyl ester, R₁₂ is a substituted or unsubstituted ring system selected from the group consisting of a C₃-C₁₁ carbocyclic ring system and a C₃-C₁₁ heterocyclic ring system; or R₁₁, and R₁₂ together form a substituted or unsubstituted ring system selected from the group consisting of a C₃-C₁₁ carbocyclic ring system and a C₃-C₁₁ heterocyclic ring system.

19. The pharmaceutical composition of claim 18, wherein R₁₀ is H or a branched C₃-C₈ alkyl.

20. The pharmaceutical composition of claim 18, wherein R₁₀ is H or t-butyl.

21. The pharmaceutical composition of claim 18, wherein t is 0 or 1.

22. The pharmaceutical composition of claim 18, wherein R₁₁ is C(O)O—C₂H₅.

23. The pharmaceutical composition of claim 18, wherein R₁₂ is a heterocyclic ring system having one or two oxygen atoms and/or one or two sulfur atoms.

24. The pharmaceutical composition of claim 18, wherein R₁₂ is

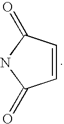

25. The pharmaceutical composition of claim 18, wherein the compound of formula III is

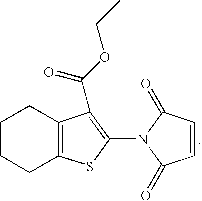

26. A method for inhibiting HIV replication in cells comprising administering to said cells a composition comprising a compound of formula III, a pharmaceutically acceptable salt thereof, or a pharmaceutically prodrug thereof and a pharmaceutically acceptable carrier, 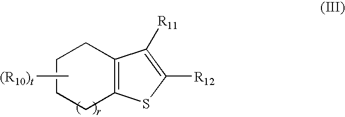
wherein:
R₁₀ is H, substituted or unsubstituted C₁-C₆ alkyl, substituted or unsubstituted C₄-C₆ cycloalkyl, or substituted or unsubstituted C₁-C₆ alkylene-C₄-C₆ cycloalkyl;
r is 1 or 2;
t is 0, 1, 2, 3, 4, or 5; and
R₁₁ is a C(O)O—(C₁-C₆) alkyl ester; R₁₂ is a substituted or unsubstituted ring system selected from the group consisting of a C₃-C₁₁ carbocyclic ring system and a C₃-C₁₁ heterocyclic ring system; or R₁₁, and R₁₂ together form a substituted or unsubstituted ring system selected from the group consisting of a C₃-C₁₁ carbocyclic ring system and a C₃-C₁₁ heterocyclic ring system.

27. Use of a compound of formula III to treat an HIV infection, comprising the step of administering to a human in need of such treatment a therapeutically effective amount of a compound of formula III 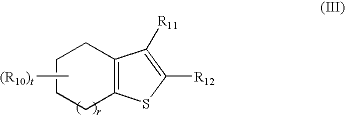
wherein:
R₁₀ is H, substituted or unsubstituted C₁-C₆ alkyl, substituted or unsubstituted C₄-C₆ cycloalkyl, or substituted or unsubstituted C₁-C₆ alkylene-C₄-C₆ cycloalkyl;
r is 1 or 2;
t is 0, 1, 2, 3, 4, or 5; and
R₁₁ is a C(O)O—(C₁-C₆) alkyl ester; R₁₂ is a substituted or unsubstituted ring system selected from the group consisting of a C₃-C₁₁ carbocyclic ring system and a C₃-C₁₁ heterocyclic ring system; or R₁₁ and R₁₂ together form a substituted or unsubstituted ring system selected from the group consisting of a C₃-C₁₁ carbocyclic ring system and a C₃-C₁₁ heterocyclic ring system.
